SEQUENCE LISTING (1 sequence)

SEQ ID NO: 1 - protein, 15 aa, Artificial Sequence
  synthetic
RMWPSSTVNLSAGRR

(19) United States
(12) Patent Application Publication
(10) Pub. No.: US 2016/0129098 A1
(43) Pub. Date: May 12, 2016

(54) TARGETING DNA VACCINES TO B CELLS AS PRIMARY ANTIGEN PRESENTING CELLS

(71) Applicant: Wisconsin Alumni Research Foundation, Madison, WI (US)

(72) Inventors: Douglas G. McNeel, Madison, WI (US); Viswa T. Colluru, Madison, WI (US)

(21) Appl. No.: 14/935,095

(22) Filed: Nov. 6, 2015

Related U.S. Application Data
(60) Provisional application No. 62/076,987, filed on Nov. 7, 2014.

(51) Int. Cl. A61K 39/00

(52) U.S. Cl. CPC A61K 39/0011; A61K 2039/53; A61K 2039/6031; A61K 2039/55555; A61K 2039/572; A61K 2039/585; A61K 2039/55522; A61K 2039/6056

(57) ABSTRACT

It is disclosed herein that B cells, not dendritic cells or myeloid-derived populations, are primary human antigen presenting cells for plasmid DNA. Based on this finding, improved methods and compositions for administering DNA vaccines are disclosed. Specifically, DNA vaccines are co-administered with a B cell targeting agent, B-cell recruiting agent, or a monocyte or dendritic cell recruiting agent. To increase the immunogenicity of the DNA vaccines, the B cell targeting agent or B cell recruiting agent is administered at the same location where the DNA vaccine is administered. In contrast, the monocyte or dendritic cell recruiting agent can be administered in a different location, in order to recruit cells competing with the B cells for DNA uptake away from the location where the DNA vaccine is administered.

### CROSS-REFERENCE TO RELATED APPLICATIONS

This application claims the benefit of U.S. provisional Application No. 62/076,987 filed Nov. 7, 2014, which is incorporated by reference herein in its entirety.

### STATEMENT REGARDING FEDERALLY SPONSORED RESEARCH OR DEVELOPMENT

This invention was made with government support under CA 142608 awarded by the National Institutes of Health. The government has certain rights in the invention.

### BACKGROUND OF THE INVENTION

An antigen is a molecule, often but not always a polypeptide, that is capable of stimulating an immune response against target cells containing the antigen. Nucleic acid-based vaccines, for example, DNA or RNA vaccines, are used to deliver DNA or RNA coding for the antigen into a cell to produce the antigen of interest and elicit an immune response. DNA vaccines can include DNA vectors (including, without limitation, naked or linear DNA, conventional plasmids, minicircle vectors, or mini-intronic plasmids) administered in vivo that encode a polypeptide antigen that is expressed by cells and elicits an immune response against the antigen. For example, in order for a DNA vaccine to specifically target tumor cells, the DNA vaccine would encode an antigen specific to or more highly expressed by the targeted tumor cells. An example of such an antigen is the ligand-binding domain of the androgen receptor (AR LBD), which is more highly expressed in prostate tumor cells than in other normal tissues, such as liver, muscle, bladder, or brain tissue.

When delivered as a vaccine, the plasmid DNA is taken up by antigen presenting cells and expressed within the antigen presenting cell to produce the antigen, which is subsequently presented to T cells to elicit a cellular immune response. Specifically, the antigens produced within the antigen presenting cell are displayed as peptide epitopes bound to major histocompatibility complex (MHC) class I and class II molecules and brought to the surface of the antigen presenting cell along with the MHC molecules. These surface antigens are then presented to immature T cells containing the transmembrane glycoprotein “cluster of differentiation 8” (CD8+ T cells) and CD4+ T cells. For example, in the case of MHC class I presentation, this can result in the activation of the immature CD8+ T cells into mature antigen-specific CD8+ T cells (also known as cytolytic T cells or killer T cells), which subsequently target and kill the cell type targeted by the vaccine. It has long been assumed that the professional antigen presenting cells doing this work are the dendritic cells (DCs), and thus most current strategies to improve the efficacy of DNA vaccines are focused on DCs.

DNA vaccines are inexpensive and safe, and pre-clinical studies have demonstrated remarkable efficacy in over 30 disease models, including those of breast, prostate and colon malignancies, multiple myeloma, lymphoma and fibrosarcoma. In spite of this, DNA vaccines have been unsuccessful in a number of human clinical trials, while achieving ‘standard of care’ status in other large animals, such as in dogs and horses.

Accordingly, a re-examination of the accepted mechanisms of plasmid DNA-induced immunity in relevant human cell systems is warranted, and there is a need in the art for improved nucleic-acid vaccines, including DNA vaccines, and methods for delivering the same resulting from an improved understanding of the mechanisms of nucleic acid-induced immunity.

### BRIEF SUMMARY

This disclosure is based on the discovery that human B cells, and not dendritic cells or myeloid-derived populations, serve as the primary antigen presenting cells for antigens coded by plasmid DNA. Specifically, the inventors have shown that delivery of DNA directly to B cells can augment antigen-specific CD8+ T cell production in mice and in a human priming system. Furthermore, the spontaneous uptake of DNA in B cells appears to be limited by the presence of larger, more phagocytic cells, such as macrophages and dendritic cells (DCs), which are able to outcompete B cells for DNA uptake. Moreover, some of these populations also express immunosuppressive cytokines following DNA uptake. Thus, using nucleic acid-based vaccines, including DNA vaccines that are specifically targeted to B cells, recruiting B cells to the site of nucleic acid-based vaccination, or recruiting competing macrophages and dendritic cells away from the site of nucleic acid-based vaccination, can greatly increase the efficiency and extent of antigen-specific CD8+ T cell activation against a target cell type resulting from nucleic acid-based vaccination. Such methods work by increasing uptake of the nucleic acid-based vaccine including DNA vaccines by the antigen presenting B cells and/or decreasing competitive uptake of the nucleic acid-based vaccine by other cell types that do not act as antigen presenting cells.

Accordingly, in a first aspect, the disclosure encompasses a method for activating antigen-specific CD8+ T cells against a target cell in a human subject. In some embodiments, the method includes the step of administering to the subject an effective amount of a nucleic acid-based vaccine comprising a polynucleotide encoding an antigen and a B cell targeting agent, whereby uptake of the polynucleotide by B cells is increased relative to uptake or expression of the polypeptide in the absence of the B cell targeting agent. In some embodiments, the polynucleotide is DNA. In other embodiments, the polynucleotide is RNA. In some embodiments, the method includes the steps of (a) administering to the subject an effective amount of a nucleic acid-base vaccine comprising a polynucleotide encoding an antigen, and (b) co-administering to the subject a B cell recruiting agent at the same location where the vaccine is administered, whereby uptake of the polynucleotide by B cells is increased relative to uptake of the polypeptide in the absence of the B cell recruiting agent; or co-administering to the subject a monocyte or dendritic cell recruiting agent at a different location from where the vaccine is administered, whereby uptake of the polynucleotide by the B cells is increased relative to uptake in the absence of the monocyte or dendritic cell recruiting agent.

In some embodiments, the polynucleotide is in a plasmid vector. As used herein, the term “plasmid vector” is not limited to conventional plasmid vectors, but also encompasses, without limitation, “minicircle vectors” that are engineered to delete the majority of the plasmid backbone, “mini-intronic plasmids” (MIPS), wherein the entire backbone of the plasmid is placed within an intron upstream of the region coding for the antigen, or linear pieces of DNA.

In some embodiments, the polynucleotide is an RNA, for example, mRNA. In some embodiments, the RNA may be complexed with protamine to protect it from RNase. In some embodiments, the RNA content is optimized to stabilize the RNA In some embodiments, the nucleotides may be modified to protect the RNA from RNAses.

In some embodiments, the antigen is the cancer-testis antigen synovial sarcoma X breakpoint-2 (SSX2), the ligand-binding domain of the androgen receptor (AR LBD), prostate-specific antigen (PSA), prostatic acid phosphatase (PAP), or human epidermal growth factor receptor 2 (HER-2/neu).

In some embodiments, the target cell is a cancer cell, including, without limitation, a prostate cancer cell, a malignant melanoma cell, a colon cancer cell, a liver cancer cell, a lung cancer cell, an ovarian cancer cell, a renal cancer cell, a pancreatic cancer cell, or a breast cancer cell.

In some embodiments, the B cell recruiting agent is a B cell chemoattractant. A non-limiting example of a B cell chemoattractant that could be used in the method is B cell attracting chemokine 1 (BCA-1, also designated CXCL-13).

In some embodiments, the B cell targeting agent includes a CD19 or CD21 targeting antibody or peptide. In some embodiments where a CD19 targeting antibody is used, the CD19 targeting antibody may be coupled to a nanoparticle, lipid-based carrier molecule, or extracellular vesicle that is complexed with the polynucleotide. In some embodiments where a CD21 targeting peptide is used, the CD21 targeting peptide includes the amino acid sequence RMWPSSTVNLSAGRR (SEQ ID NO:1). In some embodiments using a CD19 or CD21 targeting peptide, the peptide is linked to a DNA carrier. A non-limiting example of a DNA carrier that could be used in the method is protamine. In some embodiments, the extracellular vesicle is an exosome.

In a second aspect, the disclosure encompasses a nucleic acid-based vaccine, for example a DNA vaccine, for activating antigen-specific CD8+ T cells against a target cell in a human. The vaccine includes (a) a polynucleotide encoding an antigen, and (b) a B cell targeting agent, a B cell recruiting agent, or both.

In some embodiments, the polynucleotide is in a plasmid vector.

In some embodiments, the antigen is the cancer-testis antigen synovial sarcoma X breakpoint-2 (SSX2), the ligand-binding domain of the androgen receptor (AR LBD), prostate-specific antigen (PSA), human epidermal growth factor receptor 2 (HER-2/Neu) or prostatic acid phosphatase (PAP).

In some embodiments, the target cell is a cancer cell, including, without limitation, a prostate cancer cell, a malignant melanoma cell, a colon cancer cell, a liver cancer cell, a lung cancer cell, an ovarian cancer cell, a renal cancer cell, a pancreatic cancer cell, or a breast cancer cell.

In some embodiments, the B cell recruiting agent is a B cell chemoattractant. A non-limiting example of such a B cell chemoattractant is B cell attracting chemokine 1 (BCA-1, also known as CXCL-13).

In some embodiments, the B cell targeting agent is an exosome or other extracellular vesicle that increases delivery of nucleic acids to B lymphocytes. In some embodiments, this could include exosomes or extracellular vesicles that harbor B lymphocyte binding agents on their surface (including, but not limited to, protein, peptide or glycolipid molecules). In some embodiments, this could include exosomes containing the CD21 binding glycoprotein-350/220 (gp350) on their surface and transfected with a nucleic acid vaccine.

In a third aspect, the disclosure encompasses a composition that includes (a) a polynucleotide encoding an antigen, and (b) a B cell targeting agent, a B cell recruiting agent, or both, for the manufacture of a medicament for activating antigen-specific CD8+ T cells against a target cell type in a human.

In some embodiments, the polynucleotide is DNA. In some embodiments, the polynucleotide is in a plasmid vector.

In other embodiments, the polynucleotide is RNA.

In some embodiments, the antigen is the cancer-testis antigen synovial sarcoma X breakpoint-2 (SSX2), the ligand-binding domain of the androgen receptor (AR LBD), prostate-specific antigen (PSA), human epidermal growth factor receptor 2 (HER-2/neu) or prostatic acid phosphatase (PAP).

In some embodiments, the target cell is a cancer cell, including, without limitation, a prostate cancer cell, a malignant melanoma cell, a colon cancer cell, a liver cancer cell, a lung cancer cell, an ovarian cancer cell, a renal cancer cell, a pancreatic cancer cell, or a breast cancer cell.

In some embodiments, the B cell recruiting agent is a B cell chemoattractant. A non-limiting example of such a B cell chemoattractant is B-cell attracting chemokine 1 (BCA-1, also known as CXCL-13).

In some embodiments, the B cell targeting agent includes a CD19 or CD21 targeting antibody or peptide. In some embodiments including a C19 targeting antibody, the C19 targeting antibody is coupled to a nanoparticle, lipid-based carrier molecule, or extracellular vesicle that is complexed with the polynucleotide. In some embodiments including a CD21 targeting peptide, the peptide includes the amino acid sequence RMWPSSTVNLSAGRR (SEQ ID NO:1). In some embodiments, an non-limiting example of the extracellular vesicle is an exosome.

In some embodiments including a CD19 or CD21 targeting peptide, the targeting peptide is linked to a DNA carrier. A non-limiting example of a DNA carrier that could be used is protamine.

In a fourth aspect, the disclosure encompasses a method for making a nucleic acid-based vaccine for activating antigen-specific CD8+ T cells against a target cell in a human subject. The method includes the step of combining a polynucleotide encoding an antigen with a B cell targeting agent. In some embodiments, the antigen is the cancer-testis antigen synovial sarcoma X breakpoint-2 (SSX2), the ligand-binding domain of the androgen receptor (AR LBD), prostate-specific antigen (PSA), human epidermal growth factor receptor 2 (HER-2/neu) or prostatic acid phosphatase (PAP). In some embodiments, the nucleic acid-based vaccine is a DNA vaccine and the polynucleotide is DNA.

In some embodiments, the B cell targeting agent includes a CD19 or CD21 targeting antibody or peptide. In some embodiments where a CD19 targeting antibody is used, the CD19 targeting antibody may be coupled to a nanosphere that is complexed with the polynucleotide. In some embodiments where a CD21 targeting peptide is used, the CD21 targeting peptide includes the amino acid sequence RMWPSSTVNLSAGRR (SEQ ID NO:1). In some embodiments using a CD19 or CD21 targeting peptide, the peptide is linked to a DNA carrier. A non-limiting example of a DNA carrier that could be used in the method is protamine. In some embodiments, the targeting antibody may be coupled to an extracellular vesicle, for example an exosome. In other embodiments, exosomes may be coupled to the targeting peptide.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 shows plasmid DNA (pDNA) uptake by human PBMC. Thawed human PBMC were co-incubated with plasmid DNA labeled with fluorescent peptide nucleic acid probe (PNA-pDNA) and sorted by FACS. Top left panel: No DNA control. Top right panel: PNA-DNA Sorted cells were stained with fluorescent markers for different cells and analyzed by flow cytometry. Bottom left panel: 13.5% of plasmid positive events were CD19+. Bottom right panel: 66.6% of plasmid positive events were CD11c+. All of the primary human APCs exhibited rapid and spontaneous uptake of plasmid DNA.

FIG. 2 shows representative images of three antigen presenting cell types (from top panel to bottom panel: CD19+, CD11c+CD14+, and CD11c+) after plasmid has been transferred into the cell. PNA-pDNA was coincubated with human PBMC for 12 h, stained with surface markers for different APC subsets and analyzed on the Amnis ImageStream X® instrument.

FIG. 3 shows that Human B cells spontaneously produce mRNA transcripts of transferred DNA. Negatively selected APC subsets from PBMCs of 2 patients (left panel: Patient #1; right panel: Patient #2) were incubated with pEGFPc1 for 24 h, washed and subjected to RNA extraction. Levels of EGFP transcript were assayed by qRT-PCR.

FIG. 4 shows that human B cells serve as antigen presenting cells of plasmid-encoded antigen in vitro. Different cell subsets were enriched using StemSep® PE selection and incubated with T-lymphocytes from an HLA-A2⁺ patient known to have CD8+ T cells specific for HLA-A2-restricted p41 and p103 SSX2-specific epitopes. These cells were then treated with either vector alone (pTVG4) or vaccine (pTVG4-SSX2) along with 0.5 ng/mL IL-1β and 10 U/mL IL-2 for 7 days after which tetramer staining was performed. The numbers indicate the % of tetramer-positive cells among CD3+CD8+ T cells detectable after culture. Tetramer staining identifies the T cells present that are specific for the encoded antigen. The data shown demonstrate that using CD19+ B Cells, not CD11c+ dendritic cells or CD14+ monocytes/macrophages, produce significant increases in numbers of mature antigen-specific CD8+ T cells.

FIG. 5. Plasmid DNA was labeled with a Cy5 dye (Minis) and incubated for 6 hours with human PBMC, and then labeled with multiple cell surface markers. The cells with DNA uptake were gated as CD11c+ by CD19+ staining Numbers show the percentage of cells with plasmid+ uptake.

FIG. 6. CD19+ and CD11c+ cells were separated by magnetic bead separation, which allows cells to be separated by incubating the cells with magnetic nanoparticles coated with antibodies against the surface antigens characteristic of a given cell type, and cultured for 4-18 hours with Cy5-labeled plasmid DNA. Images of subcellular localization resulting from cell uptake were taken using an Amnis IMAGESTREAM™ cytometer. Shown are two representative CD19+ and CD11c+ cells with plasmid-specific uptake.

FIG. 7 CD19+, CD14+ and CD11c+ cells were separated by magnetic bead separation and cultured with CD8+ cells and DNA encoding SSX2 or vector alone (pTVG4) for 7 days. Cultures were then assessed for the frequency of SSX2-specific CD8+ T cells specific for each of the HLA-A2-specific SSX2 epitopes (p41 and p103).

FIG. 8. CD19+ cells were enriched by magnetic bead selection from C57Bl/6 mice, and cultured for 18 hours in the presence of DNA encoding AR LBD (pTVG-AR). Cells were then washed and injected intradermally into naïve syngeneic mice (n=5). Splenocytes were collected 2 weeks later and assessed for antigen-specific immune response by intracellular cytokine staining using purified AR LBD protein (AR) or ovalbumin (negative control) as stimulator antigens. Shown are the % of CD8+ T cells expressing IFNγ.

FIG. 9. Human PBMC were cultured with 100 μg/mL GMP-grade plasmid DNA (or media only) in the presence of 20 ng/mL IFNγ for 42 hours, and then assessed for IDO production by intracellular cytokine staining and flow cytometry.

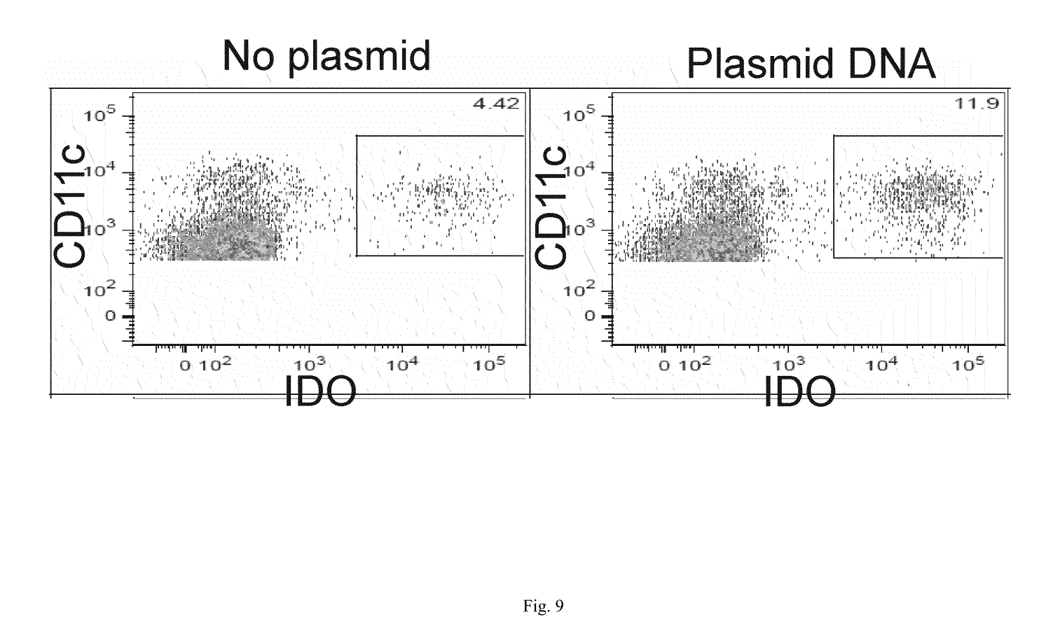

FIG. 10. 4×10⁶ human PBMC that were depleted of CD14+ cells were co-incubated with 4 μg of Cy5-labeled plasmid DNA alone, or complexed with 40 μg protamine peptide, or 40 μg CD21-protamine peptide. After 1 hour, cells were stained for CD19, and the presence of CD19+Cy5+ cells was determined by flow cytometry.

FIG. 11A. Bar graphs depicting intracellular cytokine staining for CD137 using p41 or p103 HLA-A2 restricted epitopes from SSX2 or PMA-lonomycin (positive control). CD19+ (B cells) and CD11c+ (DC) cells were enriched by magnetic bead selection from spleens of HHD-II mice, and cultured for 18 hours in the presence of plasmid DNA encoding SSX2 (pSSX2) or the p103 peptide. Cells were then washed and injected i.d. into naïve syngeneic mice (n=6 per group). Splenocytes were collected 2 weeks later, stimulated with SSX2 peptides in vitro, and assessed for antigen-specific IFNγ or IL-2 release from CD8+ T cells by intracellular cytokine staining using p41 or p103 HLA-A2 restricted epitopes from SSX2, p811 (negative control peptide) or PMA-Ionomycin (positive control). The expression of CD137 (as a marker of T cell activation) among CD8+ T cells was directly determined by flow cytometry.

FIG. 11B. Bar graphs depicting intracellular cytokine staining for IFNγ using p41 or p103 HLA-A2 restricted epitopes from SSX2 or PMA-lonomycin (positive control) after cells were pooled group-wise, expanded for 1 week with SSX2 peptides and re-assayed for Ag specific responses.

FIG. 11C. Bar graphs depicting intracellular cytokine staining for IL2 using p41 or p103 HLA-A2 restricted epitopes from SSX2 or PMA-lonomycin (positive control) after cells were pooled group-wise, expanded for 1 week with SSX2 peptides and re-assayed for Ag specific responses.

FIG. 12A. Line graph depicting average tumor size over time in mice implanted with syngeneic sarcoma cells expressing SSX2 which were subsequently immunized at bi-weekly intervals with either CD19+ (B cells) or CD11+ (DC) cells that were cultured in the presence of DNA encoding SSX2 (pTVG-SSX2) or p41/p103 peptides.

FIG. 12B. Line graph depicting tumor size over time in mice implanted with syngeneic sarcoma cells expressing SSX2 and immunized at bi-weekly intervals with CD11+ (DC) cells that were cultured in the presence of DNA encoding SSX2 (pTVG-SSX2).

FIG. 12C. Line graph depicting tumor size over time in mice implanted with syngeneic sarcoma cells expressing SSX2 and immunized with CD19+ (B cells) that were cultured in the presence of DNA encoding SSX2 (+pTVG-SSX2). CR=complete response (no tumor growth).

FIG. 12D. Line graph depicting tumor size over time in mice implanted with syngeneic sarcoma cells expressing SSX2 and immunized at bi-weekly intervals with CD11+ (DC) cells that were cultured in the presence of p41/p103 peptides.

FIG. 13A. Flow cytometery plots depicting EBV (Epstein Barr Virus) infected LCL (Lymphoblastic Cell Line) derived exosomes increased delivery of plasmid DNA to B cells in human PBMC. Whole PBMC were cultured in the presence of media only ((FIG. 13A, left), PNA-labeled DNA ((FIG. 13A, middle), or PNA-labeled DNA used to transfect exosomes derived from an EBV transformed cell line (right, FIG. 13A) for 1 hour. Cells were the assessed by flow cytometry for DNA uptake (APC+) in specific populations (CD19+ B cells, top, CD11c+CD14− DC middle, or CD14+CD11− monocytes bottom).

FIG. 13B. Graph depicting specificity of B cell uptake. Each data symbol represents a different patient across treatment conditions (naked DNA or exosome transfected DNA from two different LCLs). Uptake ratio=% plasmid positive B lymphocytes/% plasmid positive myeloid APCs.

FIG. 13C. Graph depicting % plasmid positive B cells at 24 hours. Absolute percentages of plasmid positive B lymphocytes at t=24 h.

FIG. 13D. Graph depicting exosomes cause a greater quantum of plasmid DNA to be delivered to any given B cell than incubation with naked DNA alone. Plotted are plasmid associated MFIs for upon co-incubation with naked pDNA or exosomes transfected with pDNA.

FIG. 14A. Graph depicting exosome mediated delivery of pDNA results in upregulation of CD80 on CD19+ B cells.

FIG. 14B. Graph depicting exosome mediated deliver results in upregulation of CD86 on CD19+ B cells.

FIG. 15A. Bar graph depicting exosome-pSSX2 expansion of tetramer+CD8 T cells in a patient. Whole PBMC (rather than cell subsets as in FIG. 7) were cultured in the presence of exosomes only, plasmid DNA encoding SSX2 only, or SSX2 DNA transfected exosomes derived from an EBV-transformed cell line as in FIG. 7 above. Cultures were then assessed after 7 days for the frequency of SSX2-specific (p41 and p103 epitopes) CD8+ T cells by tetramer staining Shown is the % increase in tetramer+ cells over baseline.

FIG. 15B. Bar graph depicting an increase in SSX2 specific CD8 T cells by assaying CD137/4-1BB upregulation.

### DETAILED DESCRIPTION

This disclosure provides pharmaceutical compositions and methods that relate to the use of nucleic acid-based vaccines, including plasmid DNA vaccines for the treatment of a number of disorders. Although the model systems demonstrating the disclosed methods are directed to prostate cancer treatment using a plasmid coding for the cancer-testis antigen SSX-2, the disclosed methods are applicable to any disorder that can be prevented or treated using nucleic-acid based vaccine technology, including DNA plasmid vaccine technology.

The conventional view in the art is that dendritic cells (DCs) serve as the primary antigen presenting cells for vaccine-delivered plasmid DNA. In studies with human cells, we found that both B cells and dendritic cells (DCs) can take up plasmid DNA. However, we found that DCs do not encode the protein or present the antigen directly. Instead, we found that B cells transcribe antigen and present the antigen to T cells, and thus serve as the as the primary antigen presenting cells for vaccine-delivered plasmid DNA.

While B cells have previously been identified as able to take up and deliver DNA vaccines, our finding that they serve as primary antigen presenting cells in a human system is novel. Moreover, our finding that B cells are effectively “outcompeted” by monocyte lineage cells in terms of uptake, but that such cells do not present antigen, suggests novel approaches to increase the efficacy of nucleic acid-based vaccines by recruiting and or targeting B cells in vivo. Further, extracellular vesicles, such as exosomes, can be used with the nucleic acid-based vaccines to improve specific uptake of the nucleic acids into the B cells and increase expression and presentation of the antigen to elicit an immune response.

This finding can be used to improve the efficacy of DNA vaccines by, for example, (1) targeting nucleic acid vaccines to B cells (e.g., by lipid targeting methods or extracellular vesicles, i.e. exosomes), (2) recruiting B cells to the site of immunization (e.g., by using B cell chemokines as vaccine adjuvants), (3) using B cell promoters to target expression, (4) using agents to avoid uptake by other competing cell populations (e.g., by recruiting DC or other phagocytic cells away from the site of immunization), and/or (5) using adjuvants that specifically affect B cells to improve their uptake and presentation capacity.

Although the use of DNA vectors in DNA based vaccines is well known in the art, such technology has not previously been used together with methods of B cell targeting and recruiting, and methods of avoiding competitive uptake by other cell types, as suggested by the inventors' findings disclosed herein. Each of these methods is described in further detail below.

### A. Methods of Targeting Nucleic Acid-Based Vaccines to B Cells

A number of known methods can be used to effectively target nucleic acids, including DNA to B-cells for more efficient uptake and antigen presentation. Such methods include, without limitation, the use of antibodies, peptide ligands and/or aptamers to surface proteins expressed on B lymphocytes, directly coupled with either plasmid DNA or a formulation (including, but not limited to, DNA binding proteins or polypeptides, liposomes, extracellular vesicles, exosomes or other positively charged macromolecules used alone or in combination) that binds plasmid DNA. In certain non-limiting examples, potential targeting methods can be executed as follows: (A) conjugation of antibodies targeting CD19/CD20/CD21/CD22 or other B cells surface proteins to a nucleic acid binding polypeptide, such as a DNA binding polypeptide, for example a histone or protamine, in order to bind and deliver the nucleic acid, for example the plasmid DNA, directly to cells of interest; (B) use of a peptide that displays specific binding to a B cell surface protein (CD19/20/21/22, for example) in conjunction with DNA binding or compacting agents, such as protamine, liposomes, or extracellular vesicles (e.g. exosomes) to deliver plasmid DNA; (C) use of a B cell surface receptor ligand in combination with liposomes or other equivalent DNA binding formulations; (D) use of a B cell surface receptor ligand in combination with exosomes; or (E) use of other proteins or protein formulations (viral capsids, for example) that display specificity towards B cells, along with DNA/RNA binding formulations.

In some embodiments, lipid based carrier systems are used to target the nucleic acid-based vaccines to B lymphocytes. Lipid based carrier systems include vehicles composed of physiological lipids, such as phospholipids, cholesterol, cholesterolesters and triglycerides. Suitable lipid based carriers include, but are not limited to, for example, liposomes, solid lipid nanoparticles, lipid emulsions, oily suspensions, lipid microtubules, lipid microbubbles, or lipid microspheres.

In some embodiments, suitable liposomes may be used in combination with the B cell targeting agent to deliver the polypeptide to B cells. Liposomes are artificial spherical vesicles having at least one lipid bilayer. Suitable liposomes that can be used in the practice of the present invention are known in the art. Liposomes can be prepared by disrupting biological membranes, for example, by sonication. Liposomes may be composed of phospholipids, for example, phosphatidylcholine, eggphosphatiddylethanolamine, and the like or cholesterol.

Suitable extracellular vesicles, including exosomes may be used in combination with B cell targeting agent to deliver the polypeptide of the nucleic acid-based vaccine to B cells. Extracellular vesicles (EVs) are membranous vesicles released by a variety of cells into the extracellular microenvironment. Based on the mode of biogenesis, EVs can be classified into three broad classes (i), ectosomes or microvesicles (ii), exosomes and (iii), apoptotic bodies. Exosomes are cell-derived vesicles originating from endosomal compartments produced during the vesicular transport from the endoplasmic reticulum (ER) to the Golgi apparatus. Exosomes are released extracellularly after the multivesicular bodies are fused with the plasma membrane. Suitable sources to derive exosomes for use in the present disclosure can be from any suitable cell type known in the art. Suitable cell types include, but are not limited to, immune cells, such as B lymphocytes, T lymphocytes, dendritic cells (DCs) and the like. For example, suitable cell types may be cultured cell lines, for example, but not limited to, Lymphoblastic cell lines, Human Embryonic Kidney (HEK293) cells, primary or immortalized antigen presenting cell lines among others. Exosomes may also be isolated from physiological fluids, for example, such as plasma, urine, amniotic fluid, malignant effusions and the like. In one preferred embodiment, exosomes are isolated from cell culture medium or tissue supernatant. Suitable extracellular vesicles are described in Raposa and Stoorvogel “Extracellular vesicles: Exosomes, microvesicles, and friends,” J Cell Biol. 2013 Feb. 18; 200(4):373-83. doi: 10.1083/jcb.201211138, which is incorporated by reference in its entirety.

Suitable methods to isolate and collect exosomes from culture medium are known in the art. For example, exosomes can be prepared from cell culture or tissue supernatant by centrifugation, filtration or combinations of these methods. For example, exosomes can be prepared by differential centrifugation. Not to be bound by any one method, one method uses differential centrifugation by using low speed (<20000 g) centrifugation to pellet larger particles followed by high speed (>100000 g) centrifugation to pellet exosomes. Other methods to isolate exosomes include, size filtration using filters, gradient ultracentrifugation (for example, with sucrose gradient) or a combination of these methods.

Other potential methods of specific delivery to B lymphocytes include use of native, modified or recombinant viral capsids (virus particles or “psuedovirions”) as carriers of plasmid DNA. Some potential targeting methods that could be used are discussed in greater detail in, for example, David, S., Montier, T., Carmoy, N., Resnier, P., Clavreul, A., Mevel, M., Pitard, B., Benoit, J.-P., and Passirani, C. (2012), Treatment efficacy of DNA lipid nanocapsules and DNA multimodular systems after systemic administration in a human glioma model, J Gene Med 14, 769-775; Déas, O., Angevin, E., Cherbonnier, C., Senik, A., Charpentier, B., Levillain, J. P., Oosterwijk, E., Hirsch, F., and Dürrbach, A. (2002), In Vivo-Targeted Gene Delivery Using Antibody-Based Nonviral Vector, Human Gene Therapy 13, 1101-1114; Ding, H., Prodinger, W. M., and Kope\check{c}ek, J. (2006), Identification of CD21-Binding Peptides with Phage Display and Investigation of Binding Properties of HPMA Copolymer—Peptide Conjugates. Bioconjugate Chem. 17, 514-523; Hyodo, M., Sakurai, Y., Akita, H., and Harashima, H. (2014), “Programmed packaging” for gene delivery, Journal of Controlled Release 14, 241-247; Ye, C., Choi, J. G., Abraham, S., Shankar, P., and Manjunath, N. (2014), Targeting DNA vaccines to myeloid cells using a small peptide, Eur. J. Immunol., doi: 10.1002/eji.201445010. Each of these documents is incorporated by reference herein in its entirety.

### B. Methods of Recruiting B Cells to the Site of Immunization

A number of known methods can be used to effectively recruit B-cells to the site of immunization for more efficient uptake and antigen presentation. Such methods include, without limitation, the use of chemoattractants/cytokines that specifically are known to attract and/or activate B cells. One such method would employ the properties of CXCL13 (or BCA-1/B cell attractant-1) either in nucleic acid or protein forms; CXCL13 would be employed to prime the site of immunization and/or be co-administered with plasmid DNA vaccine of interest in order to facilitate greater interaction with B cells in vivo. Other molecules that can be used in a fashion similar to BCA-1 include, but are not limited to, secondary lymphoid tissue chemoattractant (SLC), stromal cell-derived factor 1α and sphingosine-1-phosphate. These chemokines also serve as attractants to B cells and their subsets, in varying degrees of effectiveness, and can as such be employed in combination with or in place of CXCL13

### C. Using Agents to Avoid Uptake by Other Competing Cell Populations

A number of methods can be used to effectively avoid uptake of vaccine DNA by other competing cell populations. Such methods include, without limitation, use of chemoattractants/cytokines known to specifically attract cell types, such as dendritic cells, Langerhans cells and tissue resident macrophages, that compete for available DNA. Such agents can, for example, be administered at a different site from where the DNA vaccine is administered, in order to recruit the competing cells away from the vaccination site.

One of the primary approaches to accomplishing this would be administration of Granulocyte-Macrophage Colony Stimulating Factor (GM-CSF), either in nucleic acid or protein forms prior to vaccination, at a site distant from the site of immunization. Other molecules that may be used in combination or in place of GM-CSF include, but are not limited to, macrophage inflammatory protein (MIP)-1α, 1β, 3α, fms-like tyrosine kinase ligand (Flt3L), CX3CL1, MCP-1, MCP-2, MCP-3, MCP-5 CXCL8, CXCL10, RANTES, and CCL22.

### D. Using Adjuvants to Specifically Activate B Cell Populations

Adjuvants may be used to specifically activate B cell populations, rendering them active and motile. This could be used to enhance uptake of plasmid DNA by B cells as well render them better antigen presenting cells, resulting in better adaptive immunity after targeted DNA vaccination. In addition, this could also discourage uptake by competing, less activated, cell populations. These adjuvants can be co-delivered along with DNA vaccines using targeting methods or administered along with plasmid DNA post recruitment of B cells, as described in section B above. Examples of adjuvants include, but are not limited to, ligands or stimulants of Toll Like Receptors (TLR) 1, 2, 3, 4, 5, 6, 7, 9, 10 and small peptides that display adjuvant activity. Other adjuvants include chemokines or signaling molecules, CD40 ligand, NF-Kappa B subunit p65/Rel A, or Type-1 Transactivator T bet that cause activation of B cells. Polypeptide or protein molecules may be delivered either in amino acid or nucleic acid forms.

For example, use of TLR9 activating CpG agonists can cause expansion of B cells and up-regulation of its antigen presentation machinery. A potential application of this finding is codelivery of plasmid DNA and CpG molecules along with peptide or antibody mediated targeting. Suitably, the peptide targeting may include the use of extracellular vehicles, such as exosomes, to deliver the polypeptide to B cells.

In another embodiment, use of CD40 ligand (CD40L) may be used as an activating agent to cause expansion of B cells and up-regulation of its antigen presentation machinery.

In an exemplary embodiment, TLR9 can be delivered along with CXCL13 to prime the site of immunization and activate chemotactic B cells prior to delivery of the DNA vaccine. In another exemplary embodiment, alum or emulsions can be delivered along with plasmid DNA to deliver to a site to which B cells have already been attracted. In another exemplary embodiment, signaling molecules or their active fragments can be conjugated along with plasmid DNA, for either active delivery or delivery to a site where B cell chemotaxis has been effected.

In a further exemplary embodiment, extracellular vesicles, for example, exosomes, can be used to deliver the nucleic acid, for example DNA to a site where B cells have been attracted.

As used herein, an “effective amount” or an “immunologically effective amount” means that the administration of that amount to a subject, either in a single dose or as part of a series, is effective for inducing an immune reaction and preferably for treating or preventing the targeted disorder, such as, for example, prostate cancer. A number of specific disorders may targeted by the disclosed methods and compositions, including, without limitation, every condition for which DNA vaccines have been created and successfully evaluated in preclinical studies (see, e.g., Liu et al. (2011), “DNA vaccines: an historical perspective and view to the future,” Immunol Rev. 239(1): 62-84, which is incorporated by reference herein).

Such conditions include viral infections, such as HIV, Influenza, Rabies, Hepatitis B and C, Ebola, Herpes simplex, Papilloma, CMV, Rotavirus, Measles, LCMV, St. Louis encephalitis, and West Nile virus; bacterial infections, such as B. Burgdorferi, C. Tetani, M. Tb., and S. Typhi; parasitic infections, such as malaria, mycoplasma, leishmania, Toxo. Gondii, Taenia ovis, and schistosoma; cancers, such as breast, colon, prostate, myeloma, E7-induced cancer, Lymphoma, and fibrosarcoma; allergic conditions, such as house dust mite, experimental airway hyperresponsiveness (Asthma), and peanut allergy; and autoimmune diseases, such as diabetes, and EAE (Multiple sclerosis model).

In some embodiments, “target cell type” or “target cell” is a cell expressing the specific antigen or a cell that expresses high amounts of the antigen on its surface. The target cell type can include, but is not limited to, a cancer cell, a virally infected cell, a cell infected with a bacteria, among others.

Pharmaceutically acceptable carriers may be used with the disclosed methods and compositions, and are well known to those of ordinary skill in the art (Amon, R. (Ed.) Synthetic Vaccines 1:83-92, CRC Press, Inc., Boca Raton, Fla., 1987). They include liquid media suitable for use as vehicles to introduce the compositions into a patient but should not in themselves induce the production of antibodies harmful to the individual receiving the composition. An example of such liquid media is saline solution. Moreover, the vaccine formulation may also contain an adjuvant for stimulating the immune response and thereby enhancing the effect of the vaccine. Non-limiting examples of adjuvants include conventional adjuvants, such as aluminum salts, and genetic adjuvants, such as the IL-12 gene.

The nucleic acid-based vaccines of the present disclosure, when directly introduced into mammals such as humans in vivo, induce the expression of encoded polypeptide antigens within the mammals, and cause the mammals' immune system to become reactive against the antigens. Specifically, the expressed antigens elicit antigen-specific cytotoxic T lymphocytes (CTL) immunity in an MHC class I diverse population, Antigens that may be encoded/expressed in the disclosed methods and compositions include, without limitation, those listed by M. A. Cheever et al. (2009), “The prioritization of cancer antigens: a national cancer institute pilot project for the acceleration of translational research,” Clin Cancer Res. 15(17):5323-37, which is incorporated by reference herein.

The nucleic-acid based vaccines of the present invention can be used in a prime-boost strategy to induce robust and long-lasting immune response to antigen(s) encoded by the vaccine. Priming and boosting vaccination protocols based on repeated injections of the same antigenic construct are well known and result in strong CTL responses. In general, the first dose may not produce protective immunity, but only “primes” the immune system. A protective immune response develops after the second or third dose.

In one embodiment, the nucleic acid-based vaccines of the present invention may be used in a conventional prime-boost strategy, in which the same antigen is administered to the animal in multiple doses. In a preferred embodiment, the DNA, RNA or peptide vaccine is used in one or more inoculations. These boosts are performed according to conventional techniques, and can be further optimized empirically in terms of schedule of administration, route of administration, choice of adjuvant, dose, and potential sequence when administered with another vaccine, therapy or homologous vaccine.

The invention will be more fully understood upon consideration of the following non-limiting examples. Each publication, patent, and patent publication cited in this disclosure is incorporated in reference herein in its entirety.

### Example 1

### B Lymphocyte Mediated Antigen Presentation of Plasmid DNA

In this Example, we phenotypically characterized and identified human cell subsets that exhibit in vitro spontaneous plasmid DNA uptake, synthesis of mRNA encoded by transferred plasmid DNA, and the ability to prime cytolytic T lymphocyte (CTL) responses to antigen encoded by plasmid DNA.

Methods:

Three different cell types enriched from primary human PBMC (B cells, CD19+; dendritic cells, CD11c+; and monocytes/macrophages, CD14+) were assayed for spontaneous plasmid DNA uptake, encoded mRNA production, and antigen presentation to CD8+ T cells. Plasmid DNA labeled with fluorescent peptide nucleic acid (PNA) was used to detect by fluorescence detection the uptake of plasmid DNA after co-incubation. Encoded mRNA production after co-incubation was tested using quantitative RT-PCR and flow cytometry. Antigen presentation potential of each cell type was examined using T cells from patients with known, pre-existing T cell responses to one or more tumor antigens (PAP—prostatic acid phosphatase, SSX2—synovial sarcoma breakpoint-2). The three potential antigen presenting cell (APC) subsets were enriched and co-incubated with T lymphocytes along, with either an empty vector or plasmid DNA encoding the relevant tumor antigen, and assayed for expansion of T cells after 7-10 days.

Flow Cytometry.

Frozen vials of human PBMC were washed 2× in Hank's Balanced Salt Solution (HBSS) and cultured in RPMI+10% FCS along with either the PNA-APC labeled plasmid (2 ug/mL) or no DNA (controls) for twelve hours. Cells were then washed 2× and sorted for presence of plasmid based on fluorescence in the APC channel. Cells were then spun down and stained with fluorescent CD3, CD14, CD11c and CD19 antibodies to identify cell types exhibiting fluorescence associated with plasmid DNA. Amnis Imagestream® was used for visualization.

RNA Extraction and Quantitative PCR Analysis.

RNA extraction from the three classes of PBMCs was carried out using the Rneasy Mini kit according to the manufacturer's instructions. For quantitative PCR (qPCR), RNA was collected and reverse transcribed using iScript™ cDNA Synthesis Kit (Bio-Rad, Hercules, Calif.) according to the manufacturer's instructions. qPCR was performed using SsoFast™ EvaGreen® Supermix (Bio-Rad, Hercules, Calif.) in a MyiQ™2 Two-Color Real-Time PCR Detection System (Bio-Rad, Hercules, Calif.) with an annealing temperature of 60° C. All results were analyzed by the 2^{-Δct} method relative to β-actin as a control gene.

Results:

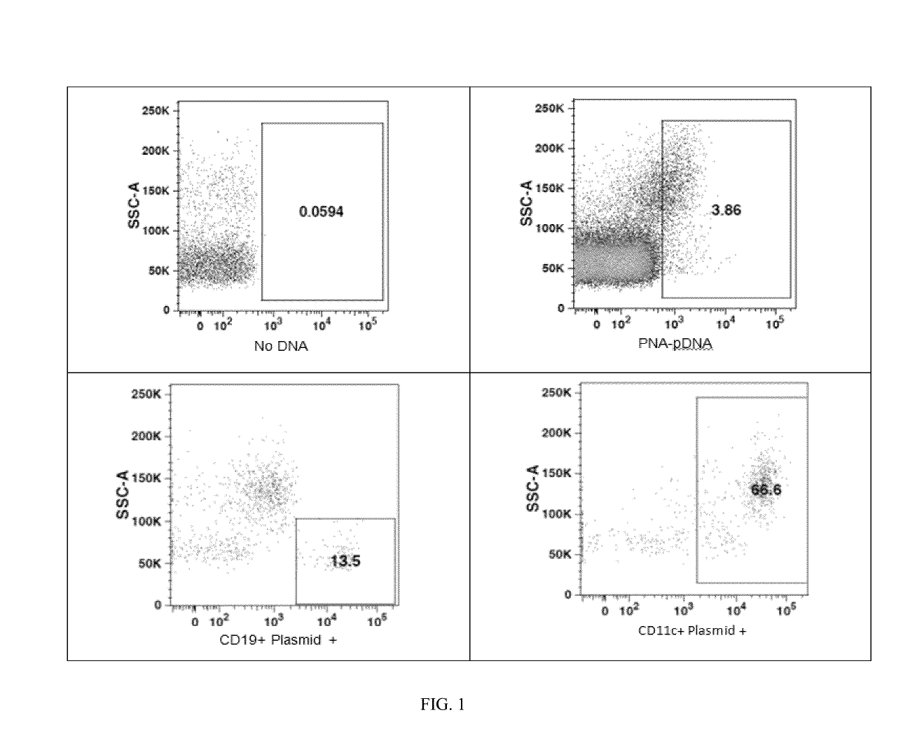

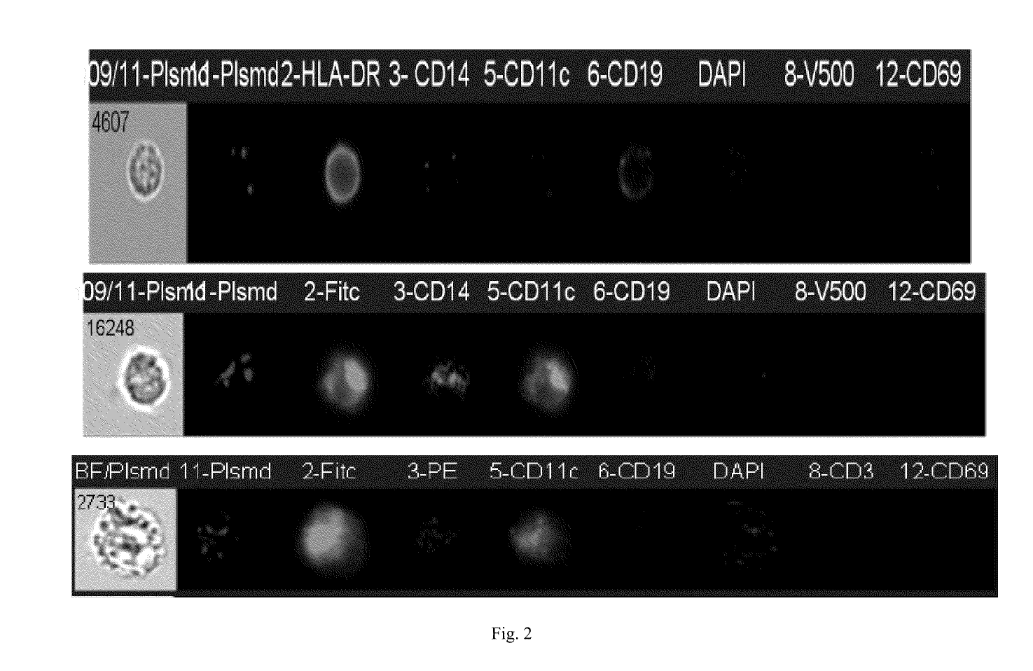

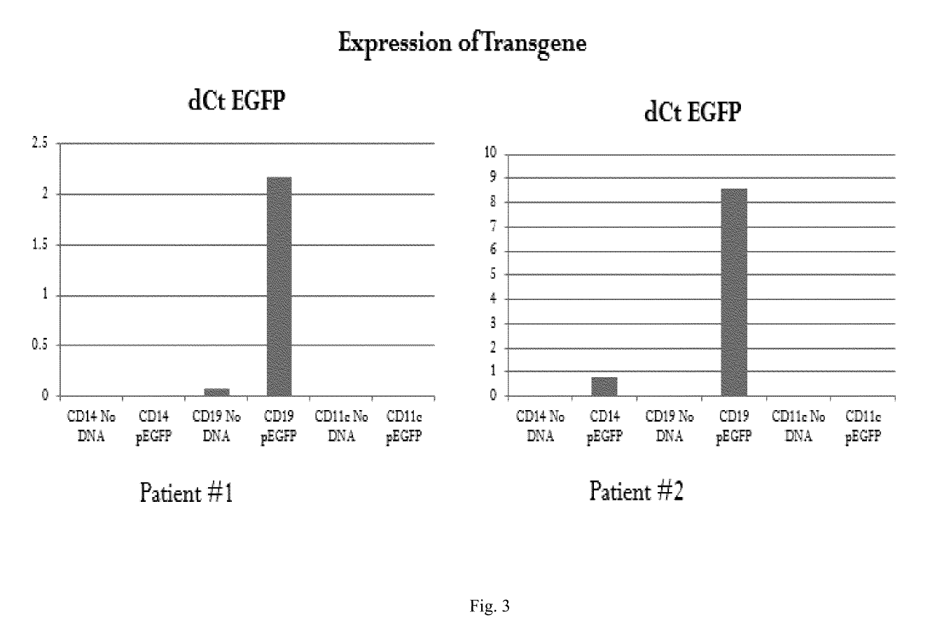

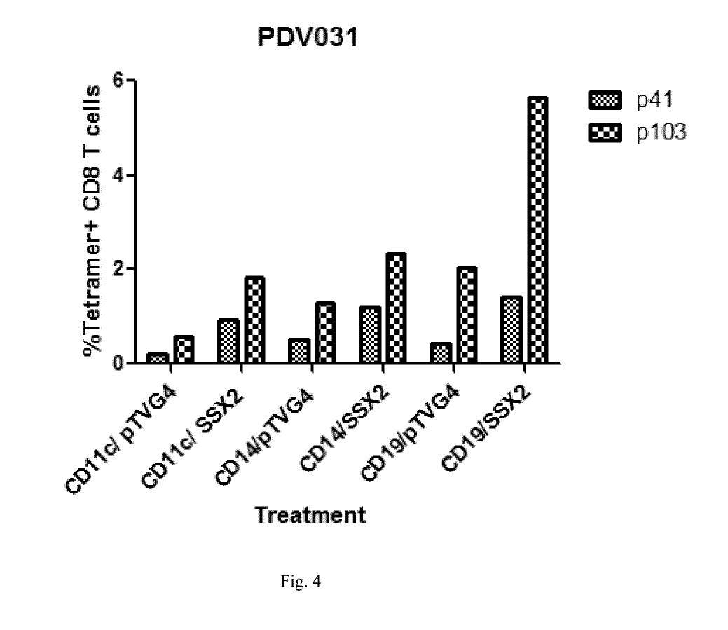

Uptake of plasmid DNA was primarily exhibited by dendritic cells (CD11c⁺, monocyte/macrophages (CD14⁺), and B lymphocytes (CD19⁺) (see FIG. 1 and FIG. 2). Plasmid uptake was verified by temperature-dependent kinetic studies and visualization of internalized plasmid by image-assisted cytometry. mRNA production was detectable only in B lymphocytes, as assessed by qRT-PCR (see FIG. 3). T lymphocytes co-incubated with B lymphocytes also displayed antigen-specific proliferation and a higher fraction of tetramer-positive CD8 T cells (see FIG. 4).

Conclusion:

Though plasmid uptake is seen in multiple human cell types, functional antigen production and presentation occurs only in specific cell subsets. These findings suggest that direct antigen presentation upon DNA vaccination is limited to B lymphocytes. Dendritic cells exhibit robust plasmid DNA uptake, but do not encode the antigen or prime immune responses, potentially playing a detrimental role in immunogenicity, because they take up DNA that is then not subsequently used to induce an immune response. Therefore, this study suggests strategies to improve DNA vaccine induced immunity in humans, such as using DNA vaccines specifically targeted to B cells, recruiting B cells to the site of DNA vaccination, or recruiting competing macrophages and dendritic cells away from the site of DNA vaccination.

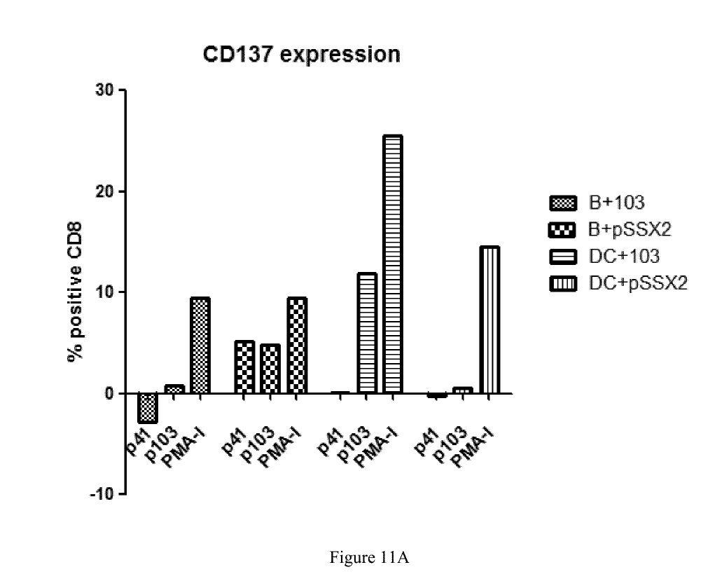

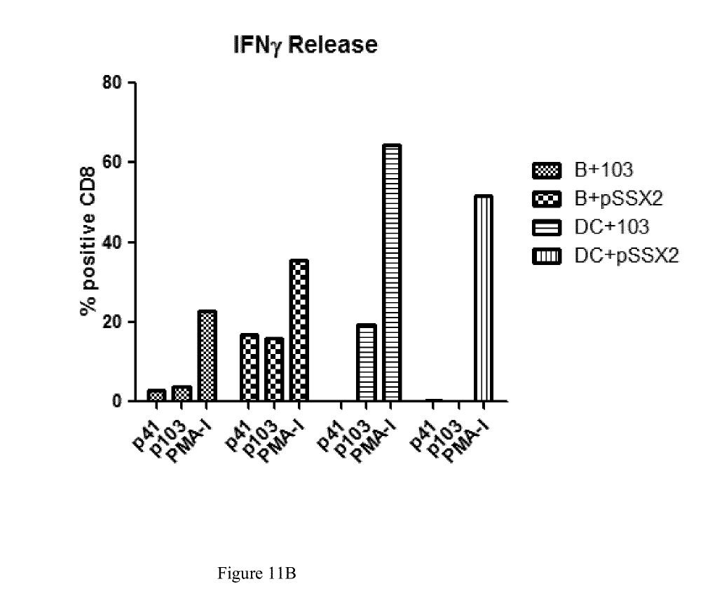

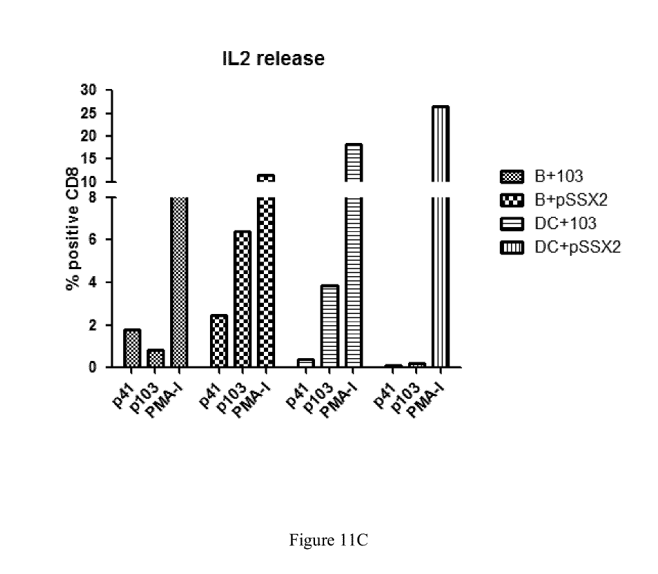

This conclusion is further supported by our data showing that CD11c+ cells (which includes macrophages/monocytes/DC) secrete IDO, an immunosuppressive cytokine, after exposure to plasmid DNA and as such might be counterproductive to the adaptive immune response induced post vaccination (see FIG. 11).

### Example 2

### B Cells are Primary Antigen Presenting Cells for Plasmid DNA

In this Example, we extend and provide further details regarding the study reported in Example 1. This Example also demonstrates the ability of B cells to serve as antigen presenting cells in vivo.

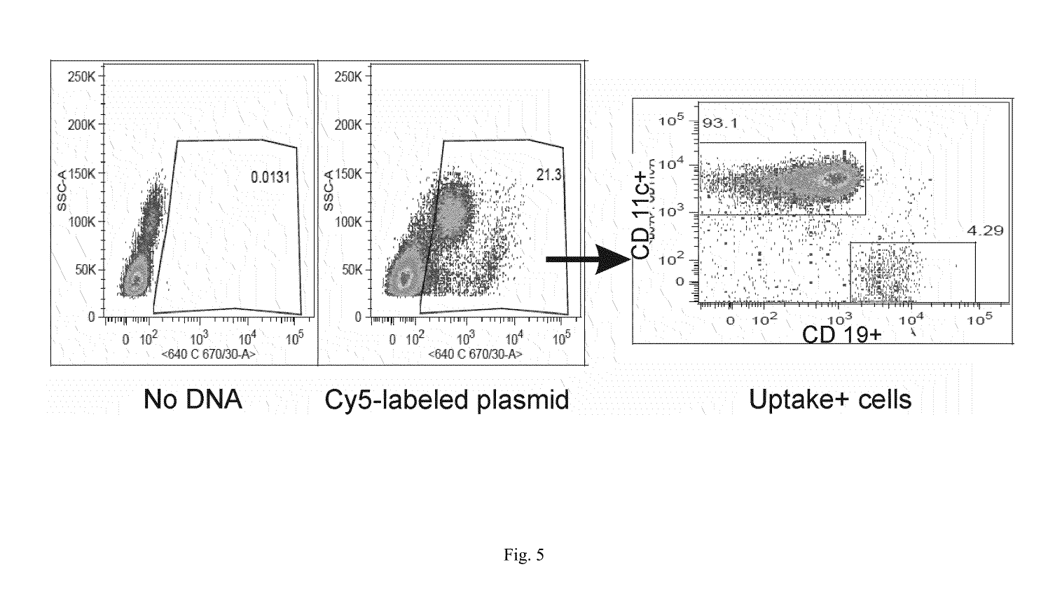

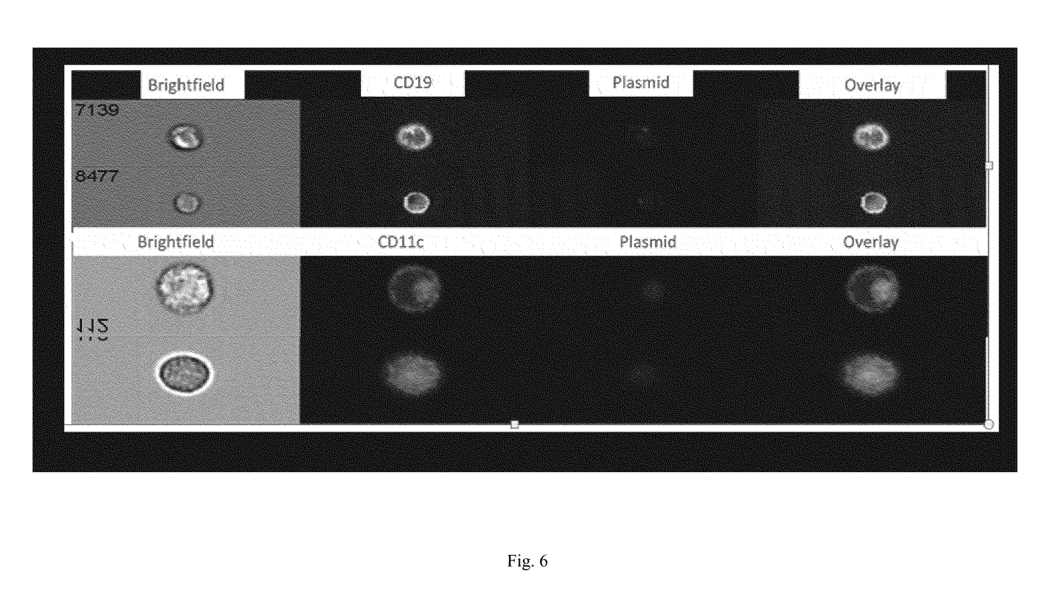

We sought to evaluate which populations of cells have the capacity for plasmid-induced primary antigen presentation, without using viruses, transfection agents, or electroporation. Peripheral blood mononuclear cells from human donors were co-cultured with plasmid DNA fluorescently labeled either with intercalating dyes or peptide-nucleic acid probes, and then evaluated for DNA uptake by each cell populations. As shown in FIG. 5, uptake (which could be competitively inhibited using unlabeled plasmid, data not shown) was predominantly by CD11c+ cells (and co-expressing CD14+, not shown) and with a minor component of uptake by CD19+ B cells. The specific B cell population responsible for plasmid uptake was subsequently identified as mature naïve B cells (CD19+IgD+). Uptake by these populations was confirmed by imaging cytometry (FIG. 6). Time course studies demonstrated that plasmid uptake occurred within a few hours, and that the plasmid was shuttled to the endosomal compartment and nucleus in B cells, and to lysosomes in DC (data not shown).

We next asked which cell population was able to encode antigen delivered by DNA. As shown in FIG. 3, magnetically sorted CD11c+, CD14+ or CD19+ cells from a single individual were co-cultured with plasmid DNA encoding GFP for 24 hours. Cells were then lysed and assayed for GFP-specific mRNA by qRT-PCR. As shown (and replicated in samples from other individuals), mRNA could only be detected following co-culture with the CD19+ B cells.

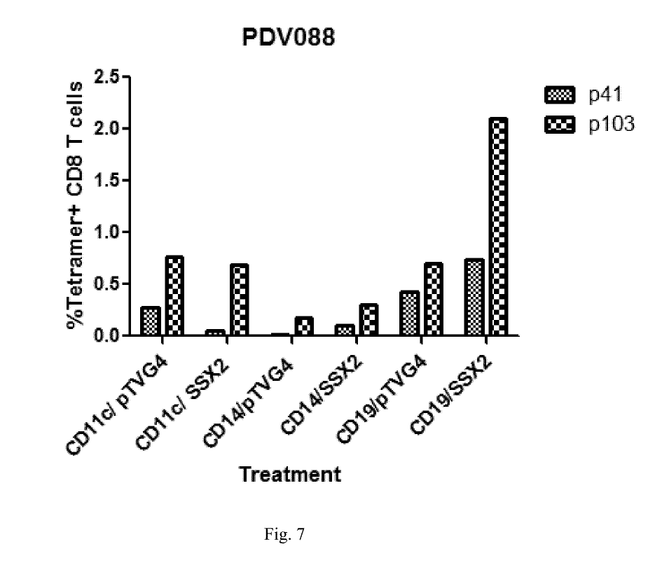

We next showed that B cells, rather than DC, can subsequently serve as antigen presenting cells. To demonstrate this, PBMC from HLA-A2+ patients with known detectable (by tetramer staining) CD8+ T cells specific for one of two epitopes derived from the antigen SSX2 (p41 epitope, or p103 epitope) (see Smith, H. A. and McNeel, D. G. (2011). “Vaccines targeting the cancer-testis antigen SSX-2 elicit HLA-A2 epitope-specific cytolytic T cells.” J Immunother 34: 569-80) were used as a source of cells. CD8+ T cells, CD11c+ cells, CD14+ cells, and CD19+ cells from the patient were separated by magnetic beads, and then each of the CD11c+ cells, CD14+ cells, and CD19+ cells were combined in three separate cultures with the CD8+ T cells (i.e., CD11c+ or CD14+ or CD19+ with CD8+ cells). Each of these three cultures was further divided into two groups, one including added plasmid DNA encoding SSX2, and the other including a vector control (pTVG4). After 1 week, each of the six cultures was assessed for the frequency of tetramer+ cells. As shown in FIG. 7 (representative from one patient, but replicated in other patient samples), CD 19+ B cells were most effective in presenting antigen and expanding the frequency of antigen-specific CD8+ T cells.

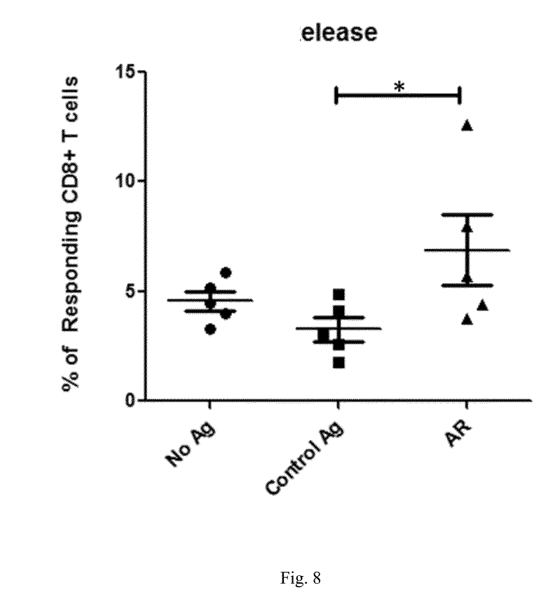

Finally, the ability of B cells to serve as antigen presenting cells was directly assessed in vivo. Specifically, B cells and DC were collected from HHD-II (HLA-A2 transgenic) mice, cultured in serum-free medium for 18 hours with plasmid DNA (encoding SSX2 or control plasmid), and then injected into syngeneic mice intradermally. Splenocytes were collected after 1 week and assessed for antigen-specific T cells by IFNγ ELISPOT. As shown in FIG. 8, B cells were found to be able to effectively present an antigen encoded by DNA directly in vivo.

Our findings demonstrate for the first time that B cells are primary human antigen presenting cells for plasmid DNA, and can expand CD8+ T cell populations in vitro. Our findings do not suggest that DC are not involved in antigen presentation following DNA administration; in fact, evidence from animal studies suggests that cross-presentation of antigen is exquisitely important (see, e.g., Akbari, O., Panjwani, N., Garcia, S., Tascon, R., Lowrie, D. and Stockinger, B. (1999). “DNA vaccination: transfection and activation of dendritic cells as key events for immunity.” J Exp Med 189: 169-78). Our findings do show, however, that targeting B cells would be particularly advantageous. Moreover, our findings suggest that the majority of DNA is taken up following human immunization by monocyte-derived cells that do not lead to a productive immune response. Hence, strategies to recruit or target B cells, while still permitting DC to cross-present antigen, would result in more effective DNA vaccine approaches.

### Example 3

### Animal Models Used to Demonstrate the Efficacy of the Disclosed Methods and Compositions

Several animal models can be used to demonstrate the efficacy of the disclosed methods and compositions. For example, our animal models include DNA vaccines encoding one of two antigens, SSX2 (a neoantigen) and the AR LBD (a “self” tolerant antigen for which the amino acid sequence is identical among different species, and which is a relevant tumor-promoting gene in prostate tumors). We have identified HLA-A2 epitopes for each antigen (see Smith, H. A. and McNeel, D. G. (2011). “Vaccines targeting the cancer-testis antigen SSX-2 elicit HLA-A2 epitope-specific cytolytic T cells.” J Immunother 34: 569-80; and Olson, B. M. and McNeel, D. G. (2011). “CD8+ T cells specific for the androgen receptor are common in patients with prostate cancer and are able to lyse prostate tumor cells.” Cancer Immunol Immunother 60: 781-92; each of which is incorporated by reference herein), and in the case of SSX2, there are just two HLA-A2 restricted CD8+ epitopes (p41 and p103), of which one (p103) is dominant. We have HLA-A2-restricted tetramer reagents specific for both antigens, permitting the studies described herein.

Moreover, one can use two related murine models. Both are derivatives of the HHD-II mouse (C57Bl/6 background), which expresses HLA-A2 and HLA-DR1 and has the murine MHC class I and class II knocked out. We have generated a methylcholanthrene (MCA) sarcoma tumor cell line from this mouse that expresses SSX2 or AR, providing a subcutaneous tumor model. In addition, we have crossed the HHD-II mouse with an autochthonous prostate tumor transgenic strain (TRAMP, in which the SV40 large T antigen is expressed downstream of the probasin prostate-specific promoter). The F1 generation expresses HLA-A2 (and murine class I), and develops prostate tumors with 100% penetrance beginning at ˜16 weeks of age (Olson, B. M., Johnson, L. E. and McNeel, D. G. (2013). “The androgen receptor: a biologically relevant vaccine target for the treatment of prostate cancer.” Cancer Immunol Immunother 62: 585-96). We have also generated a prostate tumor cell line from these mice that expresses HLA-A2 and can grow subcutaneously in syngeneic mice.

In addition to these murine models, as described in FIG. 7 (see Example 2), we have collected PBMC from multiple HLA-A2+ patients with prostate cancer, and have previously reported that SSX2+CD8+ T cells (p41- and p103-specific tetramer+ T cells) can be identified in patients with later stage prostate cancer, with some patients having as many as 1-2% of circulating CD8+ T cells specific for SSX2 (Smith, H. A. and McNeel, D. G. (2011). “Vaccines targeting the cancer-testis antigen SSX-2 elicit HLA-A2 epitope-specific cytolytic T cells.” J Immunother 34: 569-80). The availability of PMBC from multiple subjects, and the ability to use sorted cell populations in vitro to present DNA-encoded antigen (FIG. 7), effectively provides a model by which we can study human B cell uptake and presentation in vitro.

These models, in combination with other methods known in the art, provide the skilled artisan with the tools needed to make and use the disclosed compositions and methods to the full scope of this disclosure.

### Example 4

### Targeted Delivery of DNA Vaccines to B Cells to Increase the Magnitude and Effector Function of Vaccine-Elicited Antigen-Specific CD8+ T Cells

As described in the Examples above, we have identified that naïve memory B cells serve as primary antigen presenting cells for DNA vaccines. The discovery that naïve memory B cells are the primary antigen presenting cells is a novel finding in human cells, as it has been generally assumed that dendritic cells serve as primary antigen presenting cell for genetic vaccines, and many efforts have been made to improve the efficacy of DC to present antigens encoded by genetic vaccines (see, e.g., Moulin, V., Morgan, M. E., Eleveld-Trancikova, D., Haanen, J. B., Wielders, E., Looman, M. W., Janssen, R. A., Figdor, C. G., Jansen, B. J. and Adema, G. J. (2012). “Targeting dendritic cells with antigen via dendritic cell-associated promoters.” Cancer Gene Ther 19: 303-11). Our findings demonstrate that human monocyte-derived populations can also take up plasmid DNA, but cannot present encoded antigen to T cells.

Recent efforts have been directed to attempting to improve overall transfection efficiency, typically by particle-mediated delivery or by electroporation methods. While these efforts to increase cross-presentation may be useful, efforts to target DC directly may be limited. However, efforts to increase delivery to cells having the ability to directly present antigens encoded by DNA (i.e., naïve memory B cells) have been overlooked, and such an approach would either complement or vastly improve the efficacy of DNA vaccines.

As shown in FIG. 11, adoptive transfer of B cells pre-incubated with a DNA vaccine was able to elicit antigen-specific CD8+ T cells, whereas delivery of DC pre-incubated with a DNA vaccine was not. This prophetic Example shows how targeted delivery of DNA to B cells can improve the immunogenicity and anti-tumor efficacy of these vaccines.

The results reported in Examples 1 and 2 indicate that efforts to specifically target B cell uptake at the exclusion of other monocyte/DC cell subsets would be advantageous. Both of these general approaches are described below.

### A. Recruitment of B Cells to the Site of Immunization to Improve the Efficacy of DNA Vaccines.

It has been observed that GM-CSF, a chemoattractant for DC, can serve as an adjuvant for genetic vaccines, delivered either as protein or encoded by DNA (Disis, M. L., Shiota, F. M., McNeel, D. G. and Knutson, K. L. (2003). “Soluble cytokines can act as effective adjuvants in plasmid DNA vaccines targeting self tumor antigens.” Immunobiology 207: 179-86). However, our work indicates that recruitment of B cells (rather than DCs) to the site of immunization would improve the immune response elicited.

To test this, A2/TRAMP mice will receive intradermal injections of protein, or plasmid encoding, either murine GM-CSF (obtained from National Gene Vector Laboratory), as a DC chemoattractant or murine BCA-1 (B cell-attracting chemokine 1, CXCL13), as a B cell chemoattractant, or PBS alone. Animals will have biopsies taken at 6 hour intervals for up to 48 hours to identify by immunohistochemistry and flow cytometry whether B cells or DC migrate to the site of treatment, and the optimal timing for this response (time of greatest infiltration). In subsequent studies, animals pretreated with either agent (or PBS control) will then be immunized with pTVG-SSX2 or DNA vector control. After 7-14 days, splenocytes will be collected and assessed for the magnitude of antigen-specific CD8+ by tetramer staining and for effector function by intracellular cytokine staining (for epitope-specific release of IFNγ, TNFα, IL-2, IL-10, IL-4, IL-17, and granzyme B).

We expect that B cells will be recruited to the site of immunization by delivery of BCA-1, and that this will result in a greater magnitude immune response. Follow up studies will then determine whether this produces a greater anti-tumor response using the HLA-A2-restricted antigen-specific prostate and sarcoma tumor models described above, and will investigate the resulting immune responses following repetitive prime-boost immunizations using the same schedule of site priming and immunization. Because our data suggest that certain APC populations may be disadvantageous and compete for B cell uptake, a related strategy will be to attempt recruitment of these populations away from the site of immunization, for example by delivery of GM-CSF or CXCL10 (chemoattractant for monocytes) at a site away from the site of immunization.

### B. Targeted Delivery of DNA Vaccine to B Cells by Nanoparticles or Peptide-Specific Delivery to Increase Antigen-Specific Immunity.

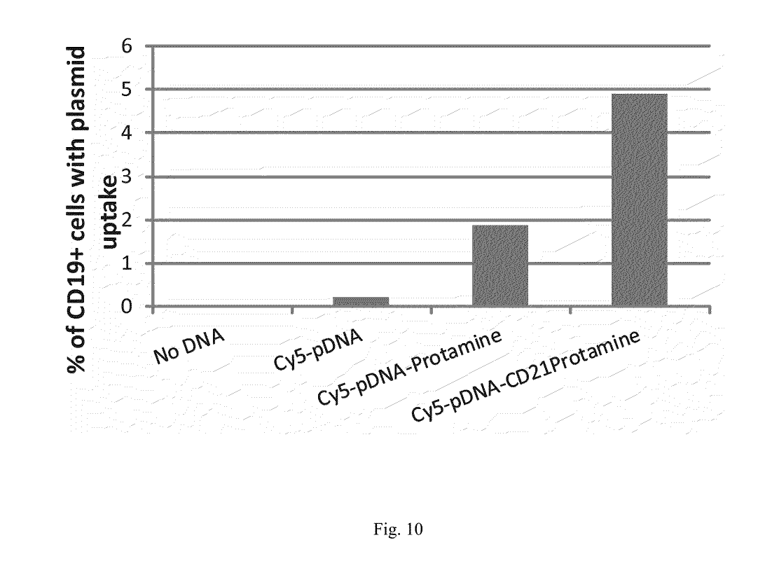

As noted above, our finding that B cells have the capacity to serve as primary antigen presenting cells suggests that they be specifically targeted. To directly target B cells in vivo, a number of methods could be used. As a non-limiting example, DNA encoding SSX2 can be complexed in nanospheres permitting direct intracellular delivery or in nanospheres coupled with antibodies to murine CD19 to target uptake to B cells. In a second non-limiting example, a CD21-targeted small peptide (RMWPSSTVNLSAGRR (SEQ ID NO:1; Ding, H., Prodinger, W. M. and Kopecek, J. (2006). “Identification of CD21-binding peptides with phage display and investigation of binding properties of HPMA copolymer-peptide conjugates.” Bioconjug Chem 17: 514-23, which is incorporated by reference herein) linked to protamine as a DNA carrier can be cultured with plasmid DNA, in a method similar to one recently reported for targeting myeloid cells (Ye, C., Choi, J. G., Abraham, S., Shankar, P. and Manjunath, N. (2014). “Targeting DNA vaccines to myeloid cells using a small peptide.” Eur J Immunol, incorporated by reference herein). FIG. 10 demonstrates that DNA conjugated with a CD21-targeted small peptide linked to protamine as a DNA carrier increased specific uptake of the DNA by B cells.

With either reagent approach, A2/TRAMP mice may be immunized once (or with a booster immunization 14 days later) by intradermal delivery of nanosphere/DNA or peptide/DNA complex (or of control plasmids containing antigen-coding DNA, but not the nanospheres or peptides). CD8+ T cells specific for SSX2 can be quantified as above by tetramer staining, and the function of these cells will be evaluated with respect to cytokine secretion by intracellular cytokine analysis. Subsequent studies will use A2/TRAMP mice implanted prior to immunization with antigen-expressing tumors, to determine whether immunization with one or the other targeted delivery approach confers a greater anti-tumor response, as compared with plasmid DNA immunization alone (the control).

Targeted delivery of plasmid DNA to B cells greatly increase the CD8+ immune response, and hence follow up studies could combine methods of B cell recruitment (such as by using BCA-1 encoding plasmid DNA to prime the site of immunization) with targeted delivery. Delivery directly to the cytoplasm of B cells by the nanosphere approach could be particularly advantageous to activate intracellular ampicillin-resistant phenotype plasmid DNA (pAMP DNA) sensors.

### Example 5

### CD21 Peptide Targeting to Deliver DNA to B Cells

In this Example, we effectively demonstrate that CD21 peptide targeting can work to deliver DNA to B cells.

Human PBMC were depleted of CD14+ cells and subsequently co-incubated with no DNA (control), with Cy5-labeled plasmid DNA alone, with Cy5-labeled plasmid complexed with protamine peptide, or with Cy5-labeled plasmid complexed with CD21/protamine. Cells from the four groups were stained for CD19, and the percentage of the CD19+ cells showing plasmid uptake was determined by flow cytometry.

The results showed much greater uptake of plasmid DNA when using the CD21/protamine complex (see FIG. 10). This indicates that targeting methods can be successfully used to increase uptake of DNA, such as vaccine DNA, to targeted B cells.

### Example 6

### B Cells Prime an Immune Response In Vivo Upon Treatment with Plasmid DNA

This Example demonstrates that B lymphocytes, and not Dendritic cells, are able to prime an immune response in vivo upon treatment with plasmid DNA. Immature CD19+ and CD11c+ cells were enriched by magnetic bead selection from spleens of A2/DR1+ mice (naïve animals for CD19+ isolation and B 16 Flt3 tumor bearing mice for CD11c+ isolation), and cultured for 18 hours in the presence of plasmid DNA encoding SSX2 (pTVG-SSX2) or the p103 peptide. Cells were then washed and injected intradermally into naïve syngeneic mice (n=6 per group). Splenocytes were collected 2 weeks later, and pooled group-wise, expanded for 1 week with SSX2 peptides and re-assayed for Ag specific response by intracellular cytokine staining using p41 or p103 HLA-A2 restricted epitopes from SSX2, or PMA-Ionomycin (positive control) (FIGS. 11A-C).

### Example 7

### B Cells Serve as Antigen Presenting Cells In Vivo

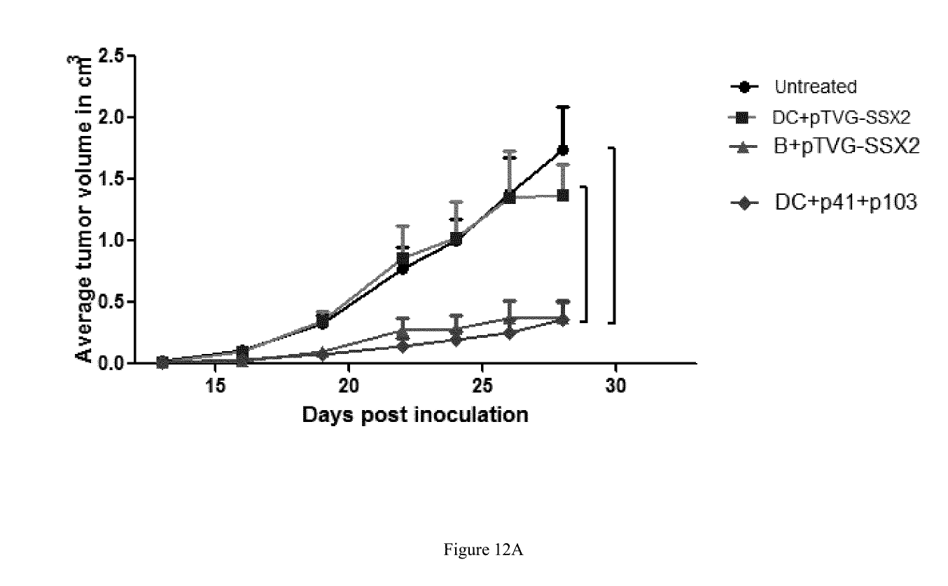

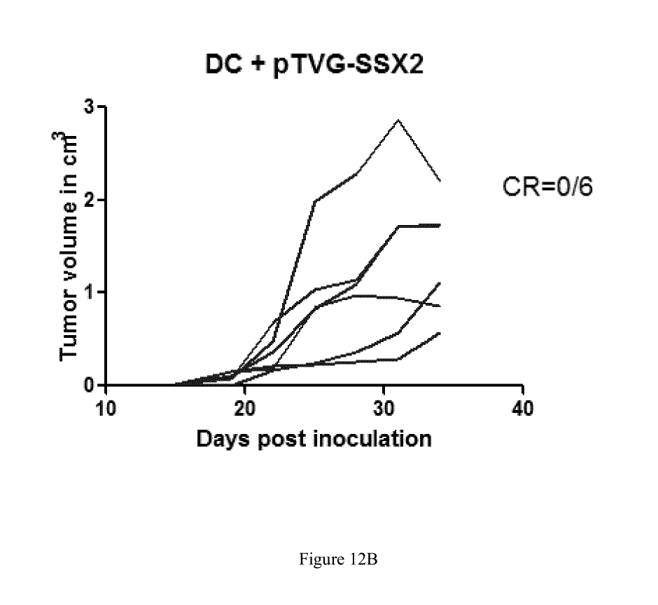

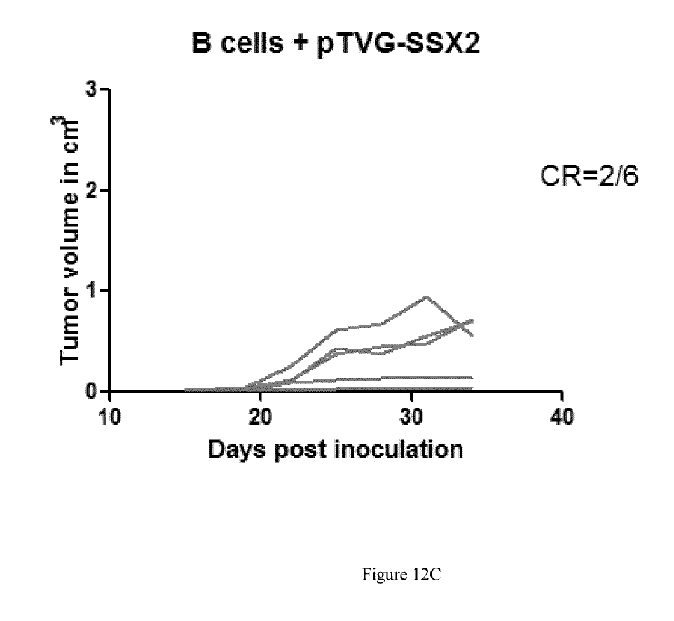

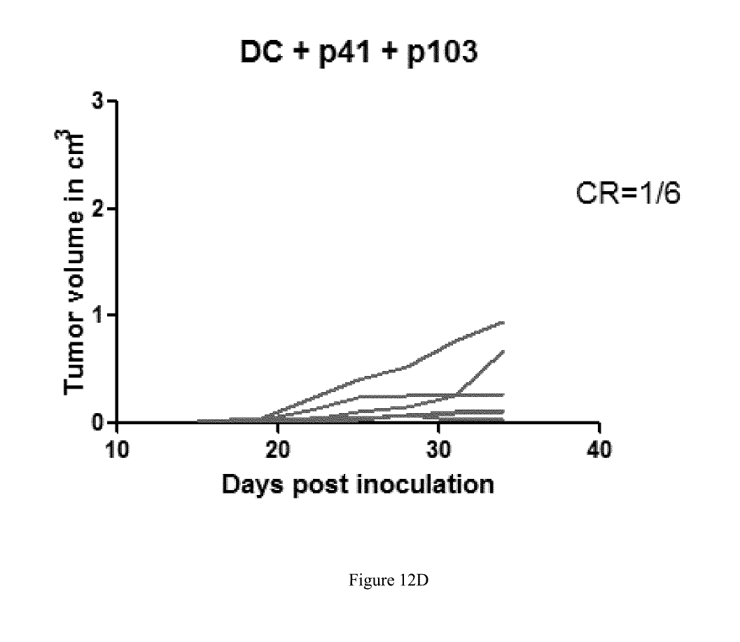

Mouse CD19+ and CD11+ cells were enriched by magnetic bead selection from A2/DR1+ mice as described in Example 6 and cultured for 18 hours at 5E6 cells/mL in the presence of 25 μg DNA encoding SSX2 (pTVG-SSX2) or 2 μg/mL p41/p103 peptides. Cells were then washed and injected intradermally at 1E6 cells/mouse into syngeneic mice that had been subcutaneously implanted 1 day prior with syngeneic sarcoma cells expressing SSX2. Mice were immunized at bi-weekly intervals, and tumor volumes measured over time. Average tumor volume is depicted in FIG. 12A, and tumor volume of mice injected with DC+pTVG-SSX2 (FIG. 12B), B cells+pTVG-SSX2 (FIG. 12C) and DC+p41+p103 (FIG. 12D) are shown.

### Example 8

### Exosomes Increase Delivery of pDNA to B Cells

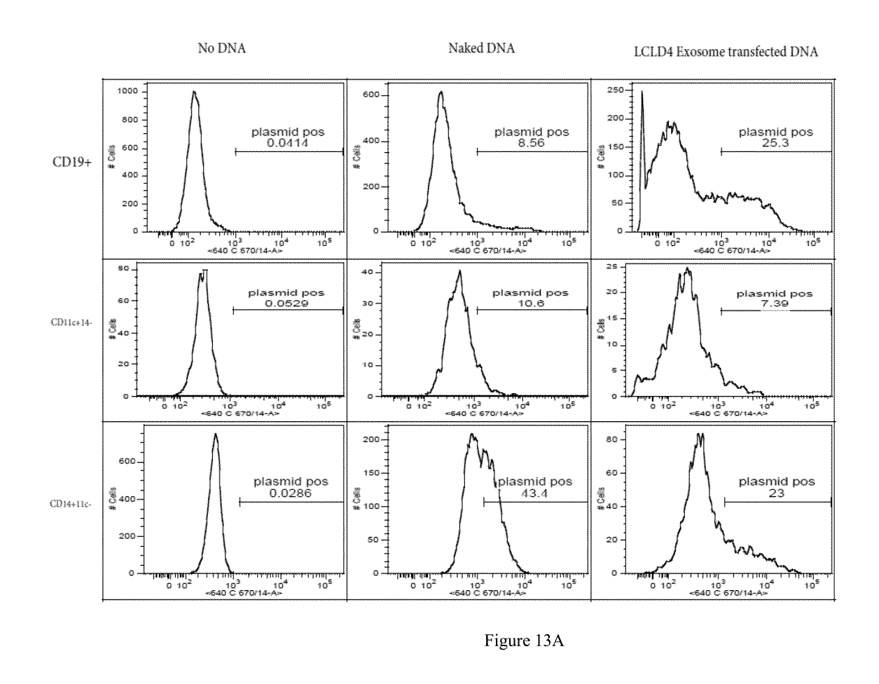

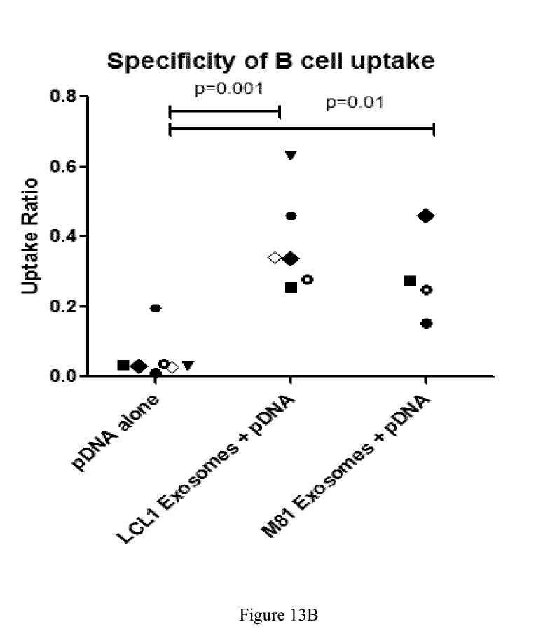

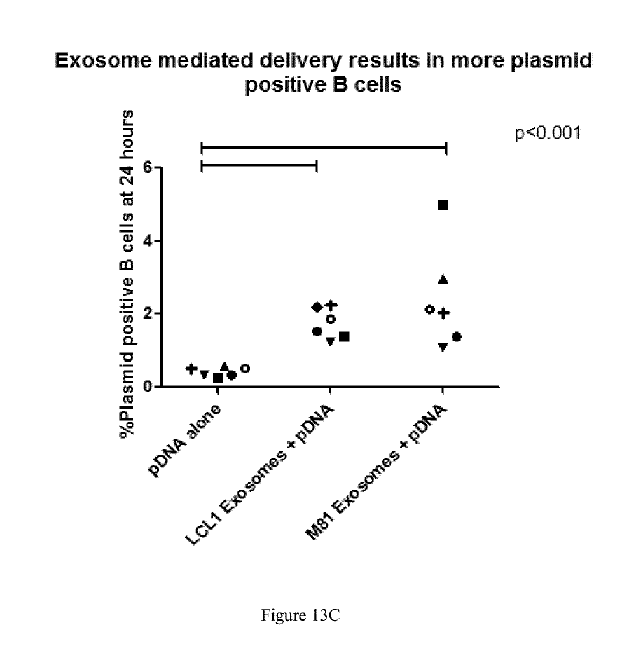

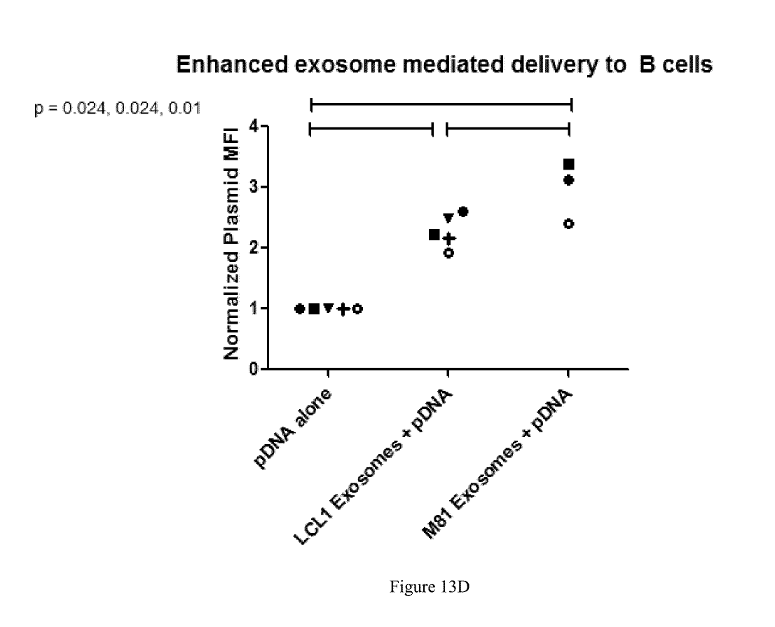

EBV (Epstein Barr Virus) infected LCL (Lymphoblastic Cell Line) derived exosomes increased delivery of plasmid DNA to B cells in human PBMC. Whole PBMC were cultured in the presence of media only ((FIG. 13A, left), PNA-labeled DNA ((FIG. 13A, middle), or PNA-labeled DNA used to transfect exosomes derived from an EBV transformed cell line (right, FIG. 13A) for 1 hour. Cells were the assessed by flow cytometry for DNA uptake (APC+) in specific populations (CD19+ B cells, top, CD11c+CD14− DC middle, or CD14+CD11− monocytes bottom). (FIGS. 13B, C, and D) Each data symbol represents a different patient across treatment conditions (naked DNA or exosome transfected DNA from two different LCLs). Uptake ratio=% plasmid positive B lymphocytes/% plasmid positive myeloid APCs (FIG. 13B). Absolute percentages of plasmid positive B lymphocytes at t=24 h (FIG. 13C).

Exosomes cause a greater quantum of plasmid DNA to be delivered to any given B cell than incubation with naked DNA alone (FIG. 13D). Plotted are plasmid associated MFIs for upon co-incubation with naked pDNA or exosomes transfected with pDNA.

### Example 9

### Exosome Mediated Delivery of Plasmid DNA to B Cells Activates Antigen Presenting Machinery on the Cell Surface

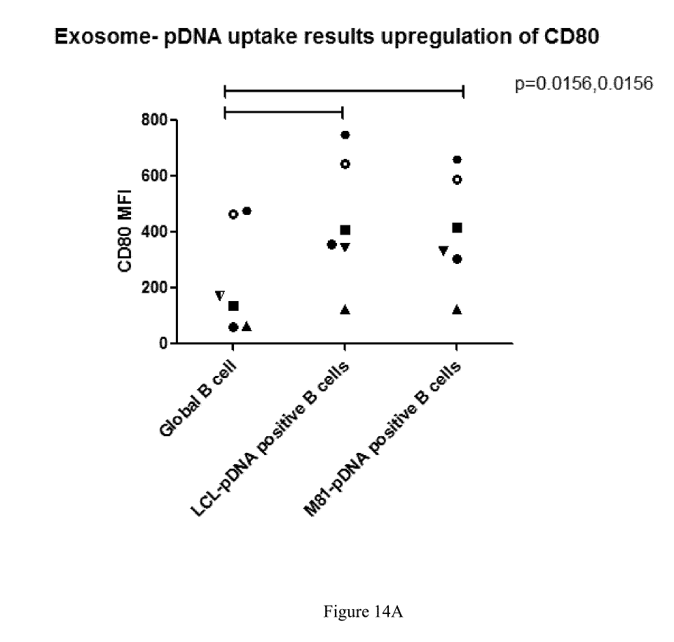

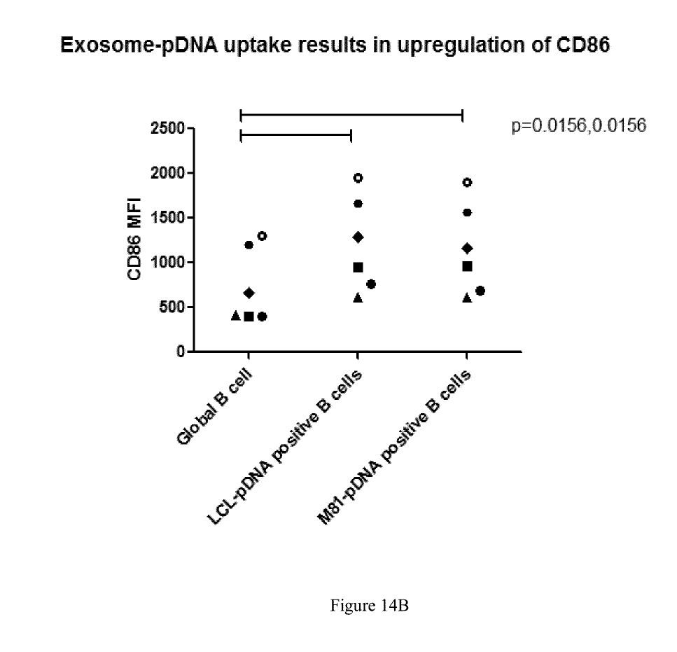

Unseparated PBMC were incubated with exosomes transfected with fluorescently labeled plasmid DNA encoding SSX2 for 24 h. B cells harboring pDNA were then assayed for upregulation of surface antigen presenting machinery markers (CD80 and CD86). Each data symbol represents a different subject under the different treatment conditions. Upregulation of surface CD80 and CD86 costimulatory molecules in B cells that are positive for exosome delivered fluorescent plasmid DNA when compared to global B cell levels in an untreated sample are shown in FIGS. 14A and B.

### Example 10

### Exosome Delivered DNA Causes Expansion of Antigen Specific T Cells

Exosomes transfected with plasmid DNA encoding SSX2 can specifically expand SSX2 specific CD8 T cells.

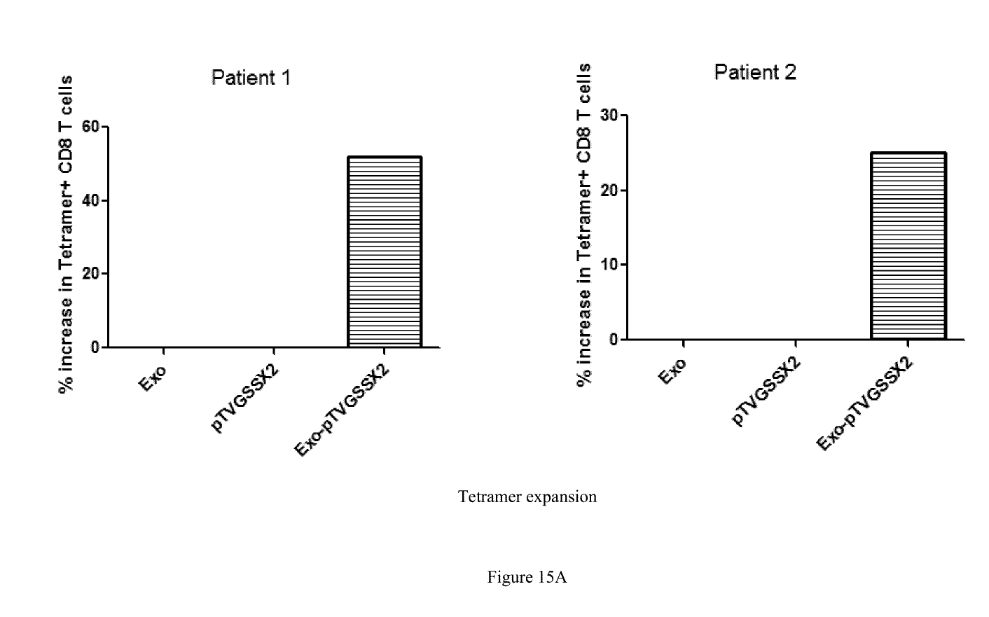

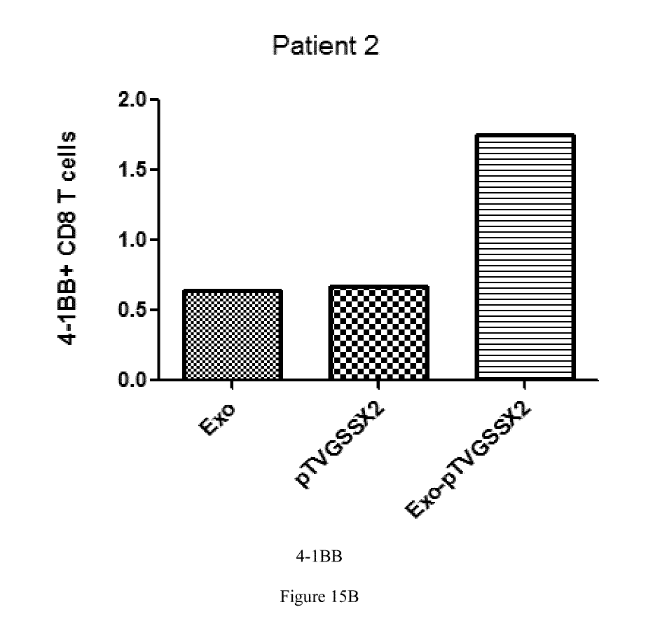

PBMC from patients with pre-existing CD8 responses to SSX2 were treated with IL2 and either exosomes alone, pTVG-SSX2 alone or exosomes transfected with pTVG-SSX2 for 1 week. Samples were then assayed for an increase in SSX2 specific CD8 T cells using HLA-A2 tetramer analysis (FIG. 15A) and CD137/4-1BB upregulation (FIG. 15B).

Each publication, patent, and patent publication cited in this disclosure is incorporated in reference herein in its entirety. The present invention is not intended to be limited to the foregoing examples, but encompasses all such modifications and variations as come within the scope of the appended claims.

We claim:

1. A method for activating antigen-specific CD8+ T cells against a target cell type in a human subject, the method comprising:
(a) administering to the subject an effective amount of a nucleic acid-based vaccine comprising a polynucleotide encoding an antigen, and a B cell targeting agent, whereby uptake of the polynucleotide by B cells is increased relative to uptake of the polypeptide in the absence of the B cell targeting agent; or
(b) administering to the subject an effective amount of a nucleic acid-based vaccine comprising a polynucleotide encoding an antigen; and
co-administering to the subject a B cell recruiting agent at the same location where the nucleic acid-based vaccine is administered, whereby uptake of the polynucleotide by B cells is increased relative to uptake or expression of the polypeptide in the absence of the B cell recruiting agent; or
(c) administering to the subject an effective amount of a nucleic acid-based vaccine comprising a polynucleotide encoding an antigen, and co-administering to the subject a monocyte or dendritic cell recruiting agent at a different location from where the nucleic acid-based vaccine is administered, whereby uptake of the polynucleotide by competing cell populations is decreased relative to uptake in the absence of the monocyte or dendritic cell recruiting agent.

2. The method of claim 1, wherein the nucleic acid-based vaccine is a DNA vaccine and the polypeptide is DNA.

3. The method of claim 1, wherein the nucleic acid-based vaccine is an RNA vaccine and the polypeptide is RNA.

4. The method of claim 1, wherein the polynucleotide is in a plasmid vector.

5. The method of claim 1, wherein the antigen is SSX2, AR LBD, PSA, HER-2/neu, or PAP.

6. The method of claim 1, wherein the B cell recruiting agent is a B cell chemoattractant.

7. The method of claim 6, wherein the B cell chemoattractant is B-cell attracting chemokine 1 (BCA-1; CXCL-13).

8. The method of claim 1, wherein the B cell targeting agent comprises a CD19 or CD21 targeting antibody or peptide.

9. The method of claim 8, wherein the B cell targeting agent comprises a CD19 targeting antibody coupled to a nanoparticle, lipid-based carrier molecule, or extracellular vesicle that is complexed with the polynucleotide.

10. The method of claim 9, wherein the lipid-based carrier molecule is a liposome or the extracellular vesicle is an exosome.

11. The method of claim 1, wherein the B cell targeting agent comprises an extracellular vesicle.

12. The method of claim 11, wherein the extracellular vesicle is an exosome.

13. The method of claim 8, wherein the CD21 targeting peptide has a sequence comprising SEQ ID NO:1.

14. The method of claim 8, wherein the CD19 or CD21 targeting peptide is linked to a DNA carrier.

15. The method of claim 14, wherein the DNA carrier is protamine.

16. The method of claim 1, wherein the target cell type is a cancer cell.

17. The method of claim 16, wherein the cancer cell is a prostate cancer cell, a malignant melanoma cell, a colon cancer cell, a liver cancer cell, a lung cancer cell, an ovarian cancer cell, a renal cancer cell, a pancreatic cancer cell, or a breast cancer cell.

18. A nucleic acid-based vaccine for activating antigen-specific CD8+ T cells against a target cell type in a human comprising:
(a) a polynucleotide encoding an antigen; and
(b) a B-cell targeting agent, a B-cell recruiting agent, or both.

19. The nucleic acid-based vaccine of claim 18, wherein the vaccine is a DNA vaccine and the polynucleotide is DNA.

20. The nucleic acid-based vaccine of claim 18, wherein the vaccine is an RNA vaccine and the polynucleotide is RNA.

21. The DNA vaccine of claim 18, wherein the polynucleotide is in a plasmid vector.

22. The nucleic acid-based vaccine of claim 18, wherein the antigen is SSX2, AR LBD, PSA, HER-2/neu or PAP.

23. The nucleic acid-based vaccine of claim 18, wherein the B cell recruiting agent is a B cell chemoattractant.

24. The nucleic acid-based vaccine of claim 18, wherein the B cell chemoattractant is B cell attracting chemokine 1 (BCA-1; CXCL-13).

25. The nucleic acid-based vaccine of claim 18, wherein the B cell targeting agent comprises a CD19 or CD21 targeting antibody or peptide.

26. The nucleic acid-based vaccine of claim 25, wherein the B cell targeting agent comprises a CD19 targeting antibody coupled to a nanoparticle, lipid-based carrier molecule, or extracellular vesicle that is complexed with the polynucleotide.

27. The nucleic acid-based vaccine of claim 25, wherein the lipid-based carrier molecule is a liposome or the extracellular vesicle is an exosome.

28. The nucleic acid-based vaccine of claim 18, wherein the B cell targeting agent comprises an exosome.

29. The nucleic acid-based vaccine of claim 25, wherein the CD21 targeting peptide has a sequence comprising SEQ ID NO:1.

30. The nucleic acid-based vaccine of claim 25, wherein the CD19 or CD21 targeting peptide is linked to a DNA carrier.

31. The nucleic acid-based vaccine of claim 30, wherein the DNA carrier is protamine.

32. The nucleic acid-based vaccine of claim 18, wherein the target cell type is a cancer cell.

33. The nucleic acid-based vaccine of claim 32, wherein the cancer cell is a prostate cancer cell, a malignant melanoma cell, a colon cancer cell, a liver cancer cell, a lung cancer cell, an ovarian cancer cell, a renal cancer cell, a pancreatic cancer cell, or a breast cancer cell.

34. A method for making a DNA vaccine for activating antigen-specific CD8+ T cells against a target cell type in a human subject, comprising combining a polynucleotide encoding an antigen with a B cell targeting agent.

35. The method of claim 34, wherein the antigen is SSX2, AR LBD, PSA, HER-2/neu or PAP.

36. The method of claim 34, wherein the B cell targeting agent is a CD19 or CD21 targeting antibody or peptide.

37. The method of claim 34, wherein the CD19 targeting antibody is used, and wherein the CD19 targeting antibody may be coupled to a nanoparticle, lipid-based carrier molecule, or extracellular vesicle that is complexed with the polynucleotide.

38. The method of claim 36, wherein the CD21 targeting peptide is used, and wherein the CD21 targeting peptide comprises the amino acid sequence RMWPSSTVNLSAGRR (SEQ ID NO:1).

39. The method of claim 36, wherein the CD19 or CD21 targeting peptide is used, and wherein the targeting peptide is linked to a DNA carrier.

40. The method of claim 39, wherein the DNA carrier is protamine.

41. The method of claim 34, wherein the B cell targeting agent comprises an exosome.
